SEQUENCE LISTING (24 sequences)

SEQ ID NO: 1 - protein, 973 aa, Homo sapiens [sequence omitted]

SEQ ID NO: 2 - protein, 866 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGDSEHKLETSSGRVLGRFRGKGSGGLNLGNFFASRKGYSRKGFDRLSTEGSDQEKEDDGSESEEEYSAPLPALAPSSS

SEQ ID NO: 3 - protein, 905 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKDSEHKLETSSGRVLGRFRGKGSGGLNLGNFFASRKGYSRKGFDRLSTEGSDQEKEDDGSESEEEYSAPLPALAPSSS

SEQ ID NO: 4 - protein, 887 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKGKGSGGLNLGNFFASRKGYSRKGFDRLSTEGSDQEKEDDGSESEEEYSAPLPALAPSSS

SEQ ID NO: 5 - protein, 974 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGADSEHKLETSSGRVLGRFRGKGSGGLNLGNFFASRKGYSRKGFDRLSTEGSDQEKEDDGSESEEEYSAPLPALAPSSS

SEQ ID NO: 6 - protein, 955 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGGKGSGGLNLGNFFASRKGYSRKGFDRLSTEGSDQEKEDDGSESEEEYSAPLPALAPSSS

SEQ ID NO: 7 - protein, 464 aa, Homo sapiens
FKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTG

SEQ ID NO: 8 - protein, 323 aa, Homo sapiens
DFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSV

SEQ ID NO: 9 - protein, 20 aa, Homo sapiens
MKWVTFISLLFLFSSAYSFR

SEQ ID NO: 10 - protein, 814 aa, Homo sapiens
SPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVGS

SEQ ID NO: 11 - protein, 15 aa, Artificial Sequence
  Peptide
CLGTTRPVVPIDSSD

SEQ ID NO: 12 - protein, 20 aa, Artificial Sequence
  Peptide
CNMPSSTGSYWFCDEGTCTD

SEQ ID NO: 13 - protein, 13 aa, Artificial Sequence
  Peptide
YGDISAFRDNNKD

SEQ ID NO: 14 - protein, 13 aa, Artificial Sequence
  Peptide
SVDTVIPAGEKVV

SEQ ID NO: 15 - protein, 14 aa, Artificial Sequence
  Peptide
CTQNVAPDMFRTIP

SEQ ID NO: 16 - protein, 20 aa, Artificial Sequence
  Peptide
TGEGRTEATHIGGTSSDEMC

SEQ ID NO: 17 - protein, 11 aa, Artificial Sequence
  Peptide
YRVHTHHLGKV

SEQ ID NO: 18 - protein, 16 aa, Artificial Sequence
  Peptide
QSPQLPQAFYPVGHPV

SEQ ID NO: 19 - protein, 28 aa, Artificial Sequence
  Peptide
RGDHVWDGNSFDSKFVYQQIGLGPIEED

SEQ ID NO: 20 - protein, 21 aa, Artificial Sequence
  Peptide
EGPVLILGRSMQPGSDQNHFC

SEQ ID NO: 21 - protein, 18 aa, Artificial Sequence
  Peptide
IDPNNAAVLQSSGKNLFY

SEQ ID NO: 22 - protein, 14 aa, Artificial Sequence
  Peptide
NGKPHFGDQEPVQG

SEQ ID NO: 23 - protein, 19 aa, Artificial Sequence
  Peptide
WGEESSGSSPLPGQFTVPH

SEQ ID NO: 24 - protein, 16 aa, Artificial Sequence
  Peptide
CFKTDTKEFVREIKHS

(19) United States
(12) Patent Application Publication
(10) Pub. No.: US 2023/0097988 A1
(43) Pub. Date: Mar. 30, 2023

(54) METHODS FOR DETERMINING PEPTIDYLGLYCINE ALPHA-AMIDATING MONOOXYGENASE (PAM) AND ITS USE FOR DIAGNOSTIC PURPOSE

(71) Applicant: PAM THERAGNOSTICS GMBH, Hennigsdorf (DE)

(72) Inventor: Andreas BERGMANN, Berlin (DE)

(73) Assignee: PAM THERAGNOSTICS GMBH, Hennigsdorf (DE)

(21) Appl. No.: 17/802,328

(22) PCT Filed: Feb. 25, 2021
(86) PCT No.: PCT/EP2021/054746

§ 371 Date: Aug. 25, 2022

(30) Foreign Application Priority Data
Feb. 26, 2020 (EP) 20159645.9

(51) Int. Cl. C12Q 1/26; G01N 33/574; G01N 33/68

(52) U.S. Cl. CPC C12Q 1/26; G01N 33/57488; G01N 33/6896; G01N 2800/2821; G01N 2333/90245

(57) ABSTRACT

The present invention is directed to a method for diagnosis or prognosis of a disease in a subject and/or predicting a risk of getting a disease or adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject.

The present invention is directed to methods for determining the level of PAM and/or its isoforms and/or fragments thereof in a bodily fluid sample, and its use for diagnostic purpose.

### STATE OF THE ART

Biologically active peptide hormones fulfill the function as signaling molecules. Most bioactive peptide hormones are synthesized from larger, inactive precursor peptides. During their biosynthesis, those peptides undergo several co- and posttranslational modifications, including cleavage of signal peptides, endoproteolytic cleavage of the precursor pro-peptides by specific endopeptidases mostly at pairs of basic residues, removal of basic residues by carboxypeptidases, formations of disulfide bonds and N- and O-glycosylation (Eipper et al. 1993. Protein Science 2(4): 489-97). More than half of the known neural and endocrine peptides require an additional modification step to gain full biological activity involving the formation of a c-terminal alpha-amide group (Guembe, et al. 1999. J Histochem Cytochem 47(5): 623-36). This final step of peptide hormone biosynthesis involves the action of the bifunctional enzyme peptidylglycine alpha-amidating monooxygenase (PAM). PAM specifically recognizes c-terminal glycine residues in its substrates, cleaves glyoxylate from the peptide's c-terminal glycine residue in a two-step enzymatic reaction leading to the formation of c-terminally alpha-amidated peptide hormones, wherein the resulting alpha-amide group originates from the cleaved c-terminal glycine (Prigge et al. 2004. Science 304(5672): 864-67). This amidation reaction takes place in the lumen of secretory granules prior to exocytosis of the amidated product (Martinez and Treston 1996. Molecular and Cellular Endocrinol 123: 113-17). Alpha-amidated peptides are for example adrenomedullin, substance P, vasopressin, neuropeptide Y, Amylin, calcitonin, neurokinin A and others. However, previously it was demonstrated that PAM can also catalyze the formation of alpha-amides from glycinated substrates of non-peptide character, e.g. N-fatty acyl-glycines, which are converted by PAM to primary fatty acid amides (PFAMs) like oleamide. The identified and purified peptidyl-glycine amidating activities were shown to be dependent on copper and ascorbate (Emeson et al. 1984. Journal of Neuroscience: 2604-13; Kumar et al. 2016. J Mol Endocrinol 56(4):T63-76; Wand et al. 1985. Neuroendocrinology 41: 482-89).

In humans, the PAM gene is located at chromosome 5q21.1 having a length of 160 kb containing 25 known exons (Gaier et al. 2014. BMC Endocrine Disorders 14). At least 6 isoforms are known to be generated by alternative splicing (SEQ ID 1-6). The PAM enzyme was found to be expressed at different levels in almost all mammalian cell types, with significant expression in airway epithelium, endothelial cells, ependymal cells in the brain, adult atrium, brain, kidney, pituitary, gastrointestinal tract and reproductive tissues (Chen et al. 2018. Diabetes Obes Metab 20 Suppl 2:64-76; Oldham et al. 1992. Biochem Biophys Res Commun 184(1): 323-29; Schafer et al. 1992. J Neurosci 12(1): 222-34).

However, the highest human PAM activity was described in the pituitary, the stalk and hypothalamus. The plasma amidating activity of healthy children below 15 years was significantly higher than that of healthy adults (Wand et al. 1985 Metabolism 34(11): 1044-52).

The precursor protein (1-973 amino acids) of the largest known PAM Isoform 1 (SEQ ID No. 1) encoded by the PAM cDNA is depicted in FIG. 1. The N-terminal signal sequence (amino acids 1-20) assures direction of the nascent PAM polypeptide into the secretory lumen of endoplasmic reticulum and is subsequently cleaved co-translationally. Afterwards the PAM-pro-peptide is processed by the same machinery used for the biosynthesis of integral membrane proteins and secreted proteins including cleavage of the pro-region (amino acids 21-30), assuring proper folding, disulfide bond formation, phosphorylation and glycosylation (Bousquet-Moore et al. 2010. J Neurosci Res 88(12):2535-45).

As depicted in FIG. 1, the PAM cDNA further encodes two distinct enzymatic activities. The first enzymatic activity is named peptidyl-glycine alpha-hydroxylating monooxygenase (PHM; EC 1.14.17.3), is an enzyme, capable of catalyzing the conversion of a C-terminal glycine residue to an alpha-hydroxy-glycine. The second activity is named peptidyl-a-hydroxy-glycine alpha-amidating lyase (PAL; EC 4.3.2.5) is an enzyme capable of catalyzing the conversion of an alpha-hydroxy-glycine to an alpha-amide with subsequent glyoxylate release. The sequential action of these separate enzymatic activities results in the overall peptidyl-glycine alpha amidating activity. The first enzymatic activity (PHM) is located directly upstream of the pro-region (within of amino acids 31-494 of isoform 1 (SEQ ID No. 7)). The second catalytic activity (PAL) is located after exon 16 in isoform 1 within of amino acids 495-817 (SEQ ID No. 8).

As depicted in FIG. 2, both activities may be encoded together within of one polypeptide as a membrane-bound protein (isoforms 1, 2, 5, 6; corresponding to SEQ ID No. 1, 2, 5 and 6) as well within of one polypeptide as a soluble protein lacking the transmembrane domain (isoforms 3 and 4; corresponding to SEQ ID No. 3 and 4). While isoforms 1, 2, 5 and 6 remain in the outer plasma membrane after fusion of secretory vesicles with the plasma membrane with subsequent endocytosis and recycling or degradation, soluble PAM isoforms lacking the TMD (isoforms 3 and 4) (amino acids 864-887) are co-secreted with the peptide-hormones (Wand et al. 1985 Metabolism 34(11): 1044-52). Furthermore, prohormone convertases may convert membrane bound PAM protein into soluble PAM protein by cleavage within the flexible region (exons 25/26) connecting PAL with the TMD during the secretory pathway (Bousquet-Moore et al. 2010. J Neurosci Res 88(12):2535-45). The PHM subunit may be cleaved from soluble or membrane bound PAM within the secretory pathway by prohormone convertases that address a double-basic cleavage-site in the exon 16 region. Furthermore, during endocytosis the full-length PAM protein may be also converted into a soluble form due to the action of alpha- and gamma secretases (Bousquet-Moore et al. 2010. J Neurosci Res 88(12):2535-45). Membrane bound PAM from late endosome can be further secreted in form of exosomal vesicles.

PHM and PAL activities, as well as the activity of the full-length PAM were determined in several human tissues and body fluids. However, the separated PHM and PAL activities in soluble forms will also lead to formation of c-terminally alpha amidated products from c-terminally glycinated substrates when allowed to perform their separate reactions in the same compartment, body-fluid or in vitro experimental setup. How the transfer of the PHM hydroxylated product to the PAL takes place is not exactly understood to date. There is evidence that the hydroxylated product is released into solution and is not directly transferred from PHM to PAL (Yin et al. 2011. PLoS One 6(12):e28679). Also not clear to date is the source of PAM in circulation.

The partial reaction of PHM is depicted in FIG. 2. PHM is a copper dependent monooxygenase responsible for stereo-specific hydroxylation of the c-terminal glycine at the alpha-carbon atom. During the hydroxylation reaction ascorbate is believed to be the naturally occurring reducing agent, while the oxygen in the newly formed hydroxyl group was shown to originate from molecular oxygen. The partial reaction of the PAL is depicted in FIG. 2. The catalytic action of PAL involves proton abstraction form the PHM-formed hydroxy-glycine by a protein-backbone derived base and a nucleophilic attack of hydroxyl-group oxygen to the divalent metal leading to a cleavage of glyoxylate and formation of a c-terminal amide.

Thus the term “amidating activity”, “alpha-amidating activity”, “peptidyl-glycine alpha-amidating activity” or “PAM activity” refers to the sequential enzymatic activities of PHM and PAL, independent of the present splice variant or mixtures of splice variants or post-translationally modified PAM enzymes or soluble, separated PHM or PAL activities or soluble PHM and membrane bound PAL or combinations of all mentioned forms leading to the formation of alpha amidated products of peptide or non-peptide character from glycinated substrates of peptide or non-peptide character. In other words, the term “amidating activity”, “alpha-amidating activity”, “peptidyl-glycine alpha-amidating activity” or “PAM activity” may be described as the sequential action of enzymatic activities located within amino acids 31 to 817 in the propeptide encoded by the human PAM cDNA, independent of present splice-variants or mixtures thereof.

PAM activity was analyzed in several human tissues and body fluids of healthy specimen or those suffering from several diseases. To summarize efforts that has been done in past:

Detection of PAM activities in human body-fluids mainly involves usage of radiolabeled synthetic tripeptides such as ¹²⁵I-D-TyrValGly, ¹²⁵I-N-acetyl-TyrValGly or comparably modified tripeptides and quantification of the amidated product due to gamma-scintillation (Kapuscinski et al. 1993. Clinical Endocrinology 39(1): 51-58; Wand et al. 1985 Metabolism 34(11): 1044-52; Tsukamoto et al. 1995. Internal Medicine 34(4): 229-32. Wand et al. 1987 Neurology 37: 1057-61. Wand et al. 1985 Neuroendocrinol 41: 482-89). Furthermore, Substance P-Gly or a truncated version Neuropeptide Y-Gly were utilized as substrates for PAM activity assays (Gether et al. 1991 Mol Cell Endocrinol 79 (1-3): 53-63; Hyyppä et al. 1990 Pain 43: 163-68; Jeng et al. 1990 Analytical Biochemistry 185(2): 213-19).

The presence of alpha-amidating activity in human circulation was initially proved by Wand et al. (Wand et al. 1985 Metabolism 34(11): 1044-52). They reported no sex differences but some variations of PAM activity in certain disease states: Plasma PAM activities were increased in hypothyroid adults as well as in patients with medullary thyroid carcinoma. The activity of PAM in tissues of medullary thyroid carcinoma, pheochromocytoma and pancreatic islet tumors were shown to be elevated suggesting increased formation of amidated peptides in endocrine tumor tissues (Gether et al. 1991 Mol Cell Endocrinol 79 (1-3): 53-63; Wand et al. 1985 Neuroendocrinol 41: 482-89).

Patients suffering from multiple endocrine neoplasia type 1 (MEN-1) and pernicious anemia showed a decreased plasma PAM activity in comparison to healthy control subjects (Kapuscinski et al. 1993. Clin Endocrinol 39(1): 51-58).

The presence of amidating activity in human cerebrospinal fluid (CSF) was shown by Wand and colleagues (Wand et al. 1985 Neuroendocrinol 41: 482-89). In patients suffering from Alzheimer's disease (AD) plasma PAM activities were shown to be unaltered when compared to healthy controls, while CSF PAM activities were significantly decreased in comparison to activities from normal specimen (Wand et al. 1987 Neurology 37: 1057-61). In addition, in WO2015/103594 the presence of PAM-Protein in CSF detected by mass spectrometry of AD-patients was proposed to be reduced compared to healthy controls. Moreover, ADM-NH₂, one of the amidated products of PAM, was shown to be reduced in patients with prevalent and incident Alzheimer's disease (WO2019/154900). However, no direct association of circulating PAM activities were reported to date being associated with prediction, diagnosis or progression of AD.

Amidating activity in CSF of patients with low back pain was analyzed using 1-12 Substance P-Gly (SP-Gly) as substrate (Hyyppä et al. 1990 Pain 43: 163-68). PAM activities of patients suffering from multiple sclerosis (MS) were shown to be increased in CSF, with a significant decrease in serum (Tsukamoto et al. 1995. Internal Medicine 34(4): 229-32; WO2010/005387). An association between plasma activity of PAM and type-2-diabetes was described in (WO2014/118634).

Even though some findings were made regarding PAM activity in human body fluids and diseases or disease progression, there is no information on PAM concentrations in human body fluids, particularly in the circulation, measured with an immunoassay. It is the surprising finding of the present invention to determine the level of PAM as the total amount or the activity of PAM in a bodily fluid of a subject for diagnosis, prognosis, prediction or monitoring of a disease or an adverse event.

### DETAILED DESCRIPTION OF THE INVENTION

Subject-matter of the present application is a method for diagnosis or prognosis of a disease in a subject and/or predicting a risk of getting a disease or an adverse event in a subject and/or monitoring a disease or an adverse event in a subject by determining the level of peptidylglycine alpha-amidating monooxygenase (PAM) and/or its isoforms and/or fragments thereof in a sample of bodily fluid of said subject,

wherein the disease in said subject is selected from the group comprising dementia, cardiovascular disorders, kidney diseases, cancer, inflammatory or infectious diseases and/or metabolic diseases, wherein the adverse event is selected from the group comprising a cardiac event, a cardiovascular event, a cerebrovascular event, a cancer, diabetes, infections, serious infections, sepsis-like systemic infections, sepsis and death due to all causes.

One embodiment of the present application relates to a method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, the method comprising the following steps:
determining the level of PAM and/or its isoforms and/or fragments thereof in a sample of bodily fluid of said subject,
comparing said determined amount to a predetermined threshold,
wherein said subject is diagnosed as having a disease if said determined amount is below or above said predetermined threshold, or
wherein an outcome of a disease is prognosticated if said determined amount is below or above said predetermined threshold, or
wherein the risk of getting a disease or an adverse event is predicted in said patient if said determined amount is below or above said predetermined threshold, or
wherein a disease or an adverse event of said subject is monitored.

One preferred embodiment of the present application relates a method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, wherein the level of PAM and/or its isoforms and/or fragments thereof is the total concentration of PAM and/or its isoforms and/or fragments thereof having at least 12 amino acids or the activity of PAM and/or its isoforms and/or fragments thereof in a sample of bodily fluid of said subject.

Another embodiment of the present application relates to a method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, wherein the activity of PAM and/or its isoforms and/or fragments thereof is selected from the group comprising the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

It is to be understood by the skilled artisan, that the PAM isoform sequences (SEQ ID No. 1 to 6) as represented in the sequence list, contain an N-terminal signal sequence (amino acid 1-20), that is cleaved off prior to secretion of the protein. Therefore, in a preferred embodiment the PAM isoform sequences (SEQ ID No. 1 to 6) and/or fragments thereof do not contain the N-terminal signal sequence.

Another embodiment of the present application relates to a method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, wherein the total concentration of PAM and/or its isoforms and/or fragments thereof having at least 12 amino acids is detected with an immunoassay.

Another specific embodiment of the present application relates to a method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, wherein the activity of PAM and/or its isoforms and/or fragments thereof is detected using a peptide-Gly as substrate.

Another preferred embodiment of the present application relates to a method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, wherein the peptide-Gly substrate is selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromdin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.

One embodiment of the present application relates to a method for diagnosis or prognosis of a disease in a subject and/or predicting a risk of getting a disease or an adverse event in a subject and/or monitoring a disease or adverse event in a subject by determining the level of PAM and/or its isoforms and/or fragments thereof in a sample of bodily fluid of said subject, wherein the PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

Another embodiment of the present application relates to a method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid of said subject, wherein the risk of getting a disease of a subject is determined, wherein said subject is a healthy subject.

Another embodiment of the present application relates to a method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid of said subject, wherein said disease is selected from the group of Alzheimer's disease, colorectal cancer and pancreatic cancer.

Another specific embodiment of the present application relates to a method for determining the level of PAM and/or isoforms and/or fragments thereof in a bodily fluid sample using an assay, wherein said assay is comprising two binders that bind to two different regions of PAM, wherein the two binders are directed to an epitope of at least 5 amino acids, preferably at least 4 amino acids in length, wherein said two binders are directed to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

A further embodiment of the present application relates a method for determining the activity of PAM and/or isoforms or fragments thereof in a bodily fluid sample of a subject comprising the steps
contacting said sample with a capture-binder that binds specifically to active full-length PAM, its isoforms and/or active fragments thereof,
separating PAM bound to said capture-binder
adding a substrate of PAM to said separated PAM
quantifying PAM activity by measuring the conversion of the substrate of PAM.

Another embodiment of the present application relates a method for determining the activity of PAM and/or isoforms and/or fragments thereof in a bodily fluid sample of a subject comprising the steps
contacting said sample with a substrate (peptide-Gly) of PAM for an interval of time at t=0 min and t=n+1 min
detecting the reaction product (alpha-amidated peptide) of PAM in said sample at t=0 min and t=n+1 min, and
quantifying the activity of PAM by calculating the difference of the reaction product between t=0 and t=n+1.

One specific embodiment of the present application relates a method, wherein the peptide-Gly substrate is selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromdin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.

Another embodiment of the present application relates to an use of antibodies for the determination of the level of PAM and/or its isoforms and/or fragments thereof, wherein said antibodies specifically bind to the sequences selected from the group of recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

Another preferred embodiment of the present application relates a kit for the determination of the level of PAM comprising one or more antibodies binding to PAM sequences selected from the group comprising recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

The object of the present invention is the provision of a method for determining the level of PAM and/or its isoforms and/or fragments thereof in a sample of bodily fluid. It is an object of the invention to provide respective assays and kits.

Another object of the invention is the provision of a method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid of said subject.

Another important embodiment of the invention is a method for diagnosis or prognosis of a disease in a subject and/or predicting a risk of getting a disease or adverse event in a subject and/or monitoring a disease or adverse event in a subject comprising:
determining the level of PAM and/or its isoforms and/or fragments thereof in a sample of bodily fluid of said subject,
comparing said determined amount to a predetermined threshold,
wherein said subject is diagnosed as having a disease if said determined amount is below or above said predetermined threshold, or
wherein an outcome of a disease is predicted if said determined amount is below or above said predetermined threshold, or
wherein the risk of getting a disease or adverse event is predicted in said patient if said determined amount is below or above said predetermined threshold, or
wherein a disease or adverse event of said subject is monitored.

Methods of determining the level of PAM are known in the art. In the context of a method for diagnosis or prognosis of a disease in a subject and/or predicting a risk of getting a disease or adverse event in a subject and/or monitoring a disease or adverse event in a subject according to the present invention either state-of-the art methods and assays may be used or the above-described methods and assays for determining the level of PAM may be used.

The threshold is pre-determined by measuring the level of PAM and/or its isoforms and/or fragments thereof in healthy controls and calculating e.g., the according 75-percentile, more preferably the 90-percentile, even more preferably the 95-percentile. The upper boarder of the 75-percentile, more preferably the 90-percentile, even more preferably the 95-percentile, defines the threshold for healthy versus diseased patients or healthy versus subjects at risk of getting a disease or subjects not at risk of getting an adverse event versus subjects at risk of getting an adverse event, if the level of said diseased subjects or subjects at risk of getting a disease or adverse event is above a threshold. The threshold is pre-determined by measuring the level of PAM and/or its isoforms and/or fragments thereof in healthy controls and calculating e.g., the according 25-percentile, more preferably the 10-percentile, even more preferably the 5-percentile. The lower boarder of the 25-percentile, more preferably the 10-percentile, even more preferably the 5-percentile, defines the threshold for healthy versus diseased patients or healthy versus subjects at risk of getting a disease or subjects not at risk of getting an adverse event versus subjects at risk of getting an adverse event, if the level of said diseased subjects or subjects at risk of getting a disease or adverse event is below a threshold. The level of PAM and/or its isoforms and/or fragments thereof may be detected as total PAM concentration and/or PAM activity. In relation to said percentiles, the lower threshold that divides between healthy and diseased patients or healthy versus subjects at risk of getting a disease or subjects not at risk of getting an adverse event versus subjects at risk of getting an adverse event by detecting the PAM activity in plasma may be between 15 and 8 μg/(L*h) or below, more preferably between 13.5 and 8 μg/(L*h) or below, even more preferred between 10.5 and 8 μg/(L*h) or below, most preferred below 8 μg/(L*h); PAM activity in serum may be between 10 and 5 μg/(L*h) or below, more preferably between 8 and 5 μg/(L*h) or below, most preferred below 5 μg/(L*h) using a PAM activity assay. In relation to said percentiles, the upper threshold that divides between healthy and diseased patients or healthy versus subjects at risk of getting a disease or subjects not at risk of getting an adverse event versus subjects at risk of getting an adverse event by detecting the PAM activity in plasma may be between 20 and 40 μg/(L*h) or above, more preferred between 25 and 40 μg/(L*h) or above, even more preferred between 30 and 40 μg/(L*h) or above, most preferred above 40 μg/(L*h); PAM activity in serum may be between 10 and 25 μg/(L*h) or above, more preferred between 15 and 25 μg/(L*h) or above, even more preferred between 20 and 25 μg/(L*h) or above, most preferred above 25 μg/(L*h) using a PAM activity assay.

The predetermined value can vary among particular populations selected, depending on certain factors, such as gender, age, genetics, habits, ethnicity or alike.

The person skilled in the art knows how to determine thresholds from conducted previous studies. The person skilled in the art knows that a specific threshold value may depend on the cohort used for calculating a pre-determined threshold that can be later-on used in routine. The person skilled in the art knows that a specific threshold value may depend on the calibration used in the assay. The person skilled in the art knows that a specific threshold value may depend on the sensitivity and/or specificity that seems to be acceptable for the practitioner.

The sensitivity and specificity of a diagnostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy) and “disease” populations (i.e. patients suffering from an infection). Depending on the particular diagnostic question to be addressed, the reference group must not be necessarily “normal”, but it might be a group of patients suffering from another disease, from which the diseased group of interest shall be differentiated. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a disease. ROC curves can be used even when test results do not necessarily give an accurate number. As long as one can rank results, one can create a ROC curve. For example, results of a test on “disease” samples might be ranked according to degree (e.g., 1=low, 2=normal, and 3=high). This ranking can be correlated to results in the “normal” population, and a ROC curve created. These methods are well known in the art (see, e.g., Hartley et al, 1982). Preferably, a threshold is selected to provide a ROC curve area of greater than about 0.5, more preferably greater than about 0.7. The term “about” in this context refers to +/−5% of a given measurement.

Once the threshold value is determined by using a previous study cohort and taking into consideration all the above-mentioned points the medical practitioner will use the pre-determined threshold for the methods of diagnosing or prognosing a disease and/or predicting a risk of getting a disease or an adverse event in a subject and/or monitoring a disease or adverse event according to the invention and will determine whether the subject has a value above or below said pre-determined threshold value in order to make an appropriate diagnosis, prognosis, prediction or monitoring.

The mentioned threshold values above might be different in other assays, if these have been calibrated differently from the assay system used in the present invention. Therefore, the mentioned threshold(s) shall apply for such differently calibrated assays accordingly, taking into account the differences in calibration. One possibility of quantifying the difference in calibration is a method comparison analysis (correlation) of the assay in question (e.g., PAM assay) with the respective biomarker assay used in the present invention by measuring the respective biomarker or it's activity (e.g., PAM) in samples using both methods. Another possibility is to determine with the assay in question, given this test has sufficient analytical sensitivity, the median biomarker level of a representative normal population, compare results with the median biomarker levels with another assay and recalculate the calibration based on the difference obtained by this comparison. With the calibration used in the present invention, samples from normal (healthy) subjects have been measured: the median plasma PAM activity was 18.4 μg/(L*h) (inter quartile range [IQR] 13.5-21.9 μg/(L*h)), the median serum PAM activity was 11.0 μg/(L*h) (inter quartile range [IQR] 8.1-13.1 μg/(L*h).

As used herein, the term “diagnosis” means detecting a disease or determining the stage or degree of a disease. Usually, a diagnosis of a disease is based on the evaluation of one or more factors and/or symptoms that are indicative of the disease. That is, a diagnosis can be made based on the presence, absence or amount of a factor which is indicative of presence or absence of the disease or disorder. Each factor or symptom that is considered to be indicative for the diagnosis of a particular disease does not need be exclusively related to the particular disease, e.g., there may be differential diagnoses that can be inferred from a diagnostic factor or symptom. Likewise, there may be instances where a factor or symptom that is indicative of a particular disease is present in an individual that does not have the particular disease.

The term “prognosis” as used herein refers to a prediction of the probable course and outcome of a clinical condition or disease, e.g., sepsis. A prognosis is usually made by evaluating factors or symptoms of a disease that are indicative of a favourable or unfavourable course or outcome of the disease. The phrase “determining the prognosis” as used herein refers to the process by which the skilled artisan can predict the course or outcome of a clinical condition or disease in a patient. The term “prognosis” does not refer to the ability to predict the course or outcome of a clinical condition or disease with 100% accuracy. Instead, the skilled artisan will understand that the term “prognosis” refers to an increased probability that a certain course or outcome will occur; that is, that a course or outcome is more likely to occur in a patient exhibiting a given clinical condition or disease, when compared to those individuals not exhibiting the clinical condition or disease.

In a specific embodiment of said method for diagnosis or prognosis of a disease in a subject and/or predicting a risk of getting a disease or adverse event in a subject and/or monitoring a disease or adverse event in a subject, said disease or is selected from the group comprising:
dementia, wherein said dementia is selected from the group comprising mild cognitive impairment (MCI), Alzheimer's disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions).
cardiovascular disorders, wherein said cardiovascular disorders may be selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and hemorrhagic stroke and transient ischemic attack) and myocardial infarction,
kidney diseases, wherein said kidney diseases may be selected from a group comprising renal toxicity (drug-induced kidney disease), acute kidney injury (AKI), chronic kidney disease (CKD), diabetic nephropathy, end-stage renal disease (ESRD),
cancer, wherein said cancer may be selected from a group comprising prostate cancer, breast cancer, lung cancer, colorectal cancer, bladder cancer, ovarian cancer, cervical cancer, skin cancer (including melanoma), stomach cancer, liver cancer, pancreatic cancer, leukemia, non-hodgkin's lymphoma, kidney cancer, esophagus cancer, pharyngeal cancer,
infectious diseases caused by infectious organisms such as bacteria, viruses, fungi or parasites, said infectious disease is selected from the group comprising SIRS, sepsis, and septic shock.
metabolic diseases selected from the group comprising diabetes type 1, diabetes type 2, metabolic syndrome.

In one embodiment of the present application said disease is dementia and said dementia is selected from the group comprising mild cognitive impairment (MCI), Alzheimer's disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions).

In a specific embodiment said dementia is Alzheimer's disease.

In one embodiment of the present application said disease is cancer and said cancer is selected from the group comprising prostate cancer, breast cancer, lung cancer, colorectal cancer, bladder cancer, ovarian cancer, cervical cancer, skin cancer (including melanoma), stomach cancer, liver cancer, pancreatic cancer, leukemia, non-hodgkin's lymphoma, kidney cancer, esophagus cancer and pharyngeal cancer.

In a specific embodiment said cancer is colorectal cancer and pancreatic cancer.

In one embodiment of the present application said disease is a cardiovascular disorder, wherein said cardiovascular disorder is selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and hemorrhagic stroke and transient ischemic attack) and myocardial infarction.

In a specific embodiment said cardiovascular disorder is heart failure (including acute and acute decompensated heart failure).

In another specific embodiment said cardiovascular disorder is stroke (including ischemic and hemorrhagic stroke and transient ischemic attack) and myocardial infarction.

In another specific embodiment said cardiovascular disorder is atrial fibrillation (AF).

In another specific embodiment of the present application said disease is SIRS, sepsis or septic shock.

In another specific embodiment of the present application said disease is diabetes type 1, diabetes type 2, metabolic syndrome.

The bodily fluid in the context of the method of the present invention maybe selected from the group of blood, serum, plasma, cerebrospinal fluid (CSF), urine, saliva, sputum, and pleural effusions. In a specific embodiment of said method said sample is selected from the group comprising whole blood, serum and plasma.

The term monitoring refers to controlling the development (detection of any changes) of a disease or pathophysiological status of a patient, e.g., risk of getting a disease or an adverse event, severity of a disease or response to a therapy.

Subject of the present invention is a method, wherein said monitoring is performed in order to evaluate the change of risk of getting a disease or adverse event, the change of severity of a disease or the response of a patient or subject to a therapy.

A specific subject matter of the present invention is a method, wherein said monitoring is performed in order to evaluate the response of said subject to preventive and/or therapeutic measures taken.

Subject matter of the present invention is a method according to the present invention, wherein said method is used in order to stratify said subjects into risk groups.

The term “risk”, as used herein, relates to the probability of suffering from an undesirable event or effect (e.g., a disease or an adverse event).

The term “enhanced level” means a level above a certain threshold level.

The term “reduced level” means a level below a certain threshold level.

An “adverse event” is defined as an event compromising the health of an individual. Said adverse event is not restricted to, but may be selected from the group comprising a cardiac event, a cardiovascular event, a cerebrovascular event, a cancer, diabetes, and death due to all causes. An adverse event includes infections, serious infections and sepsis-like systemic infections and sepsis. An adverse is not an event caused by an acute exogen induced adverse event and/or exogen induced trauma. Exogen induced trauma include those which may be induced by accidents, e.g., car accidents and are therefore excluded from the group of adverse events.

In a specific embodiment of the invention said adverse event is a cardiovascular event selected from the group comprising myocardial infarction, acute decompensated heart failure, stroke and mortality related to myocardial infarction, stroke or acute heart failure.

The risk for getting a disease or adverse event means the risk of getting said disease or event within a certain period of time. In a specific embodiment said period of time is within 10 years, or within 8 years, or within 5 years or within 2.5 years, or within 1 year, or within 6 months, or within 3 months, or within 30 days, or within 28 days.

In a specific embodiment of the invention, the “level of PAM and/or its isoforms and/or fragments thereof” is the total concentration (preferably expressed as weight/volume; w/v) of PAM and/or its isoforms and/or fragments thereof having at least 12 amino acids or the activity of PAM and/or its isoforms and/or fragments thereof comprising the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10 in a sample taken from a subject.

In the present disclosure the term “PAM” refers to the amino acid sequence of PAM isoform 1 to 6 as shown in SEQ ID No. 1 to 6. In some aspects, PAM disclosed herein has at least about 70%, at least about 75%, at least about 80%, at least about 85%, at least about 90%, at least about 95%, at least about 96%, at least about 97%, at least about 98%, or at least about 99% sequence identity to the amino acid sequence of SEQ ID No. 1 to 6.

In some aspects, said PAM is a functional fragment (i.e., PHM (SEQ ID No. 7) or PAL (SEQ ID No. 8), PAM conserving at least about 10%, at least about 20%, at least about 30%, at least about 40%, at least about 50%, at least about 60%, at least 70%, at least about 80%, or at least about 90% of the PAM activity of the corresponding full-length PAM). In some aspects, the PAM is a variant or a derivative of PAM disclosed herein.

The percentage of identity of an amino acid or nucleic acid sequence, or the term “% sequence identity”, is defined herein as the percentage of residues in a candidate amino acid or nucleic acid sequence that is identical with the residues in a reference sequence after aligning the two sequences and introducing gaps, if necessary, to achieve the maximum percent identity. In a preferred embodiment, the calculation of said at least percentage of sequence identity is carried out without introducing gaps. Methods and computer programs for the alignment are well known in the art, for example “Align 2” or the BLAST service of the National Center for Biotechnology Information (NCBI).

In a specific embodiment of the invention, an assay is used for determining the level of PAM and/or its isoforms and/or fragments thereof, wherein such assay is a sandwich assay, preferably a fully automated assay.

In one embodiment of the invention, it may be a so-called POC-test (point-of-care) that is a test technology, which allows performing the test within less than 1 hour near the patient without the requirement of a fully automated assay system. One example for this technology is the immunochromatographic test technology.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®, Ortho Clinical Diagnostics Vitros®.

In a specific embodiment of the invention, at least one of said two binders is labeled in order to be detected.

The preferred detection methods comprise immunoassays in various formats such as for instance radioimmunoassay (RIA), homogeneous enzyme-multiplied immunoassays (EMIT), chemiluminescence- and fluorescence-immunoassays, Enzyme-linked immunoassays (ELISA), Luminex-based bead arrays, protein microarray assays, and rapid test formats such as for instance immunochromatographic strip tests.

In a preferred embodiment, said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005); Hultschig et al. 2006. Curr Opin Chem Biol. 10 (1):4-10).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, fluorescein, fluorescein-isothiocyanate (FITC), IRD-700/800, Cyanine dyes, such as CY3, CY5, CY3.5, CY5.5, Cy7, xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, coumarines such as umbelliferone, benzimides, such as Hoechst 33258; Phenanthridines, such as Texas Red, Yakima Yellow, Alexa Fluor, PET, ethidiumbromide, acridinium dyes, carbazol dyes, Phenoxazine dyes, porphyrine dyes, polymethine dyes, and the like.

In the context of the present invention, chemiluminescence based assays comprise the use of dyes, based on the physical principles described for chemiluminescent materials in (Kirk-Othmer, Encyclopedia of chemical technology, 4th ed. 1993. John Wiley & Sons, Vol. 15: 518-562, incorporated herein by reference, including citations on pages 551-562). Preferred chemiluminescent dyes are acridinium esters.

As mentioned herein, an “assay” or “diagnostic assay” can be of any type applied in the field of diagnostics. Such an assay may be based on the binding of an analyte to be detected to one or more capture probes with a certain affinity. Binders that may be used for determining the level of PAM and/or its isoforms and/or fragments thereof exhibit an affinity constant to PAM and/or its isoforms and/or fragments thereof of at least 10⁷ M⁻¹, preferred 10⁸ M⁻¹, preferred affinity constant is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In the context of the present invention, “binder molecules” are molecules which may be used to bind target molecules or molecules of interest, i.e., analytes (i.e., in the context of the present invention PAM and its isoforms and fragments thereof), from a sample. Binder molecules have thus to be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain.

In a specific embodiment said binder may be selected from the group of antibody, antibody fragment or non-IgG scaffold.

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acid phosphatase, glucose phosphate dehydrogenase and so on.

In one embodiment of the invention at least one of said two binders is bound to a solid phase as magnetic particles, and polystyrene surfaces.

Subject matter of the invention is a method for determining the level of PAM and/or isoforms and/or fragments thereof in a bodily fluid sample using an assay, wherein said assay is comprising two binders that bind to two different epitopes of PAM, wherein the two binders are directed to an epitope of at least 5 amino acids, preferably at least 4 amino acids in length.

An epitope, also known as antigenic determinant, is the part of an antigen (e.g., peptide or protein) that is recognized by the immune system, specifically by antibodies. For example, the epitope is the specific piece of the antigen to which an antibody binds. The part of an antibody that binds to the epitope is called a paratope. The epitopes of protein antigens are divided into two categories: conformational epitopes and linear epitopes, based on their structure and interaction with the paratope.

A linear or a sequential epitope is an epitope that is recognized by antibodies by its linear sequence of amino acids, or primary structure and is formed by the 3-D conformation adopted by the interaction of contiguous amino acid residues. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues.

In one embodiment of the invention linear epitopes are related to following sequences of immunization peptides of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24).

In one embodiment of the invention, linear and/or conformational epitopes are related to the following sequences of PAM: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 10.

Said epitope may comprise at least 6 amino acids, preferably at least 5 amino acids, most preferred at least 4 amino acids.

In one embodiment of the invention said first and second binder binds to an epitope comprised within the following sequences of PAM: SEQ ID No. 1, SEQ ID No. 2, SEQ ID No. 3, SEQ ID No. 4, SEQ ID No. 5 and SEQ ID No. 6.

In one embodiment of the invention said first and second binder binds to an epitope comprised within the PAL subunit of PAM (SEQ ID No. 8).

In one embodiment of the invention said first and second binder binds to an epitope comprised within the PHM subunit of PAM (SEQ ID No. 7).

In one specific embodiment of the invention said first binder binds to an epitope comprised within the PAL subunit of PAM (SEQ ID No. 8) and said second binder binds to an epitope comprised within the PHM subunit of PAM (SEQ ID No. 7).

In one specific embodiment of the invention said first and second binder binds to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

Use of at least two binders for the determination of the level of PAM and/or its isoforms and/or fragments thereof, wherein said at least one binder is directed to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

Subject of the present invention is a method for determining the activity of PAM and/or isoforms and/or fragments thereof in a bodily fluid sample of a subject comprising the steps
Contacting said sample with a capture-binder that binds specifically to active full-length PAM, its isoforms and/or active fragments thereof,
Separating PAM bound to said capture-binder
Adding a substrate of PAM to said separated PAM
Quantifying PAM activity by measuring the conversion of the substrate of PAM.

In a specific embodiment of the present invention said method is an enzyme capture assay (ECA, see e.g., U.S. Pat. Nos. 5,612,186A, 5,601,986A).

In a specific embodiment of said method for determining PAM activity in a bodily fluid sample of a subject said separation step is a washing step that removes ingredients of the sample that are not bound to said capture-binder from the captured PAM and/or its isoforms and/or fragments thereof. That separation step can be any other step that separates PAM bound to said capture-binder from the ingredients of said bodily fluid sample.

One embodiment of the present invention involves a chemical assay for PAM. The assay uses a peptide substrate which reacts with PAM and/or its isoforms and/or fragments thereof to form a detectable reaction product. Alternatively, the rate of the reaction of the substrate can be monitored to determine the level of PAM and/or its isoforms and/or fragments thereof in a test sample.

Assays embodying such reagents and reactions can be performed in any suitable reaction vessel, for example, a test tube or well of a microtiter plate. Alternatively, assay devices may be developed in disposable form such as dipstick or test strip device formats which are well known to those skilled-in-the-art and which provide ease of manufacture and use. Such disposable assay devices may be packaged in the form of kits containing all necessary materials, reagents and instructions for use.

In an alternative assay embodiment, the rate at which the reaction occurs may be detected as an indication of the level of PAM and/or its isoforms and/or fragments thereof present in the test sample. For example, the rate at which the substrate is reacted may be used to indicate the level of PAM and/or its isoforms and/or fragments thereof present in the test sample. Alternatively, the rate at which the reaction product is formed may be used to indicate the level of PAM and/or its isoforms and/or fragments thereof present in the test sample.

In yet another embodiment, a capture or binding assay may be performed to determine the activity of PAM and/or its isoforms and/or fragments thereof. For example, an antibody reactive with PAM protein, but which does not interfere with its enzymatic activity, may be immobilized upon a solid phase. The test sample is passed over the immobile antibody, and PAM and/or its isoforms and/or fragments thereof, if present, binds to the antibody and is itself immobilized for detection. A substrate may then be added, and the reaction product may be detected to indicate the level of PAM and/or its isoforms and/or fragments thereof in the test sample. For the purposes of the present description, the term “solid phase” may be used to include any material or vessel in which or on which the assay may be performed and includes, but is not limited to, porous materials, nonporous materials, test tubes, wells, slides, etc.

In a specific embodiment of said method for the diagnosis or prognosis of a disease in a subject and/or predicting a risk of getting a disease or adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject said capture binder is immobilized on a surface. For the determination of PAM activity, a binder reactive with PAM and/or its isoforms and/or fragments thereof, but which does not interfere with enzymatic activity by more than 50%, preferably less than 40%, preferably less than 30%, may be immobilized upon a solid phase. To prevent inhibition of PAM the capture-binder should not bind PAM in the area around the active center and substrate binding region.

In a specific embodiment of said method for determining the level of PAM and/or its isoforms and/or fragments thereof in a bodily fluid sample of a subject said binder may be selected from the group of antibodies, antibody fragments, non-Ig scaffolds or aptamers.

Another subject of the present invention is a method for determining the activity of PAM and/or isoforms and/or fragments thereof in a bodily fluid sample of a subject comprising the steps
contacting said sample with a substrate (peptide-Gly) of PAM for an interval of time at t=0 min and t=n+1 min
detecting the reaction product (alpha-amidated peptide) of PAM in said sample at t=0 min and t=n+1 min, and
quantifying the activity of PAM by calculating the difference of the reaction product between t=0 and t=n+1.

Another subject of the present invention is a method for determining PAM activity in a bodily fluid sample of a subject comprising the steps
contacting said sample with the substrate ADM-Gly of PAM for an interval of time at t=0 min and t=n+1 min
detecting the reaction product ADM-NH₂ of PAM in said sample at t=0 min and t=n+1 min using an immunoassay, and
quantifying the activity of PAM by calculating the difference of the reaction product ADM-NH₂ between t=0 min and t=n+1 min.

The term “t=n+1 min” is a time interval, wherein n is defined as >0 min.

One embodiment of the present application relates to a kit for performing the method for diagnosis or prognosis of a disease in a subject and/or predicting a risk of getting a disease or an adverse event in a subject and/or monitoring a disease or adverse event in a subject, wherein said kit comprises at least two binders directed to recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

A specific embodiment of the present application relates to a kit for the detection of the level of PAM comprising one or more binders binding to PAM sequences selected from the group comprising recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

Another embodiment of the present application relates to a kit for performing the method for determining the activity of PAM and/or isoforms and/or fragments thereof in a bodily fluid sample of a subject, wherein said kit comprises peptide-Gly PAM as substrate, wherein said peptide-Gly is ADM-Gly.

The activity of PAM can be measured by detection of alpha-amidated peptides (peptide-amide) from their glycinated precursor peptide substrates (peptide-Gly). Nearly half of biologically active peptides terminate with a C-terminal alpha-amide (Vishvanatha et al. 2014.J Biol Chem 289(18):12404-20).

The glycinated precursor peptide substrates may be selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromdin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin, vasopressin.

In a preferred embodiment said peptide-Gly is adrenomedullin-Gly (ADM-Gly) and said peptide-amide is adrenomedullin-amide (ADM-NH₂).

Other substrates of non-peptide character may comprise N-fatty acyl-glycines, which are converted by PAM to primary fatty acid amides (PFAMs) like oleamide.

With the above context, the following consecutively numbered embodiments provide further specific aspects of the invention:
1. A method for diagnosis or prognosis of a disease in a subject and/or predicting a risk of getting a disease or an adverse event in a subject and/or monitoring a disease or an adverse event in a subject by determining the level of peptidylglycine alpha-amidating monooxygenase (PAM) and/or its isoforms and/or fragments thereof in a sample of bodily fluid of said subject, wherein the disease in said subject is selected from the group comprising dementia, cardiovascular disorders, kidney diseases, cancer, inflammatory or infectious diseases and/or metabolic diseases, wherein the adverse event is selected from the group comprising a cardiac event, a cardiovascular event, a cerebrovascular event, a cancer, diabetes, infections, serious infections, sepsis-like systemic infections, sepsis and death due to all causes.
2. A method for diagnosis or prognosis of a disease in a subject and/or predicting a risk of getting a disease or an adverse event in a subject and/or monitoring a disease or adverse event in a subject by determining the level of peptidylglycine alpha-amidating monooxygenase (PAM) and/or its isoforms and/or fragments thereof in a sample of bodily fluid of said subject, the method comprising the following steps:
   determining the level of PAM and/or its isoforms and/or fragments thereof in a sample of bodily fluid of said subject,
   comparing said determined amount to a predetermined threshold,
   wherein said subject is diagnosed as having a disease if said determined amount is below or above said predetermined threshold, or
   wherein an outcome of a disease is prognosticated if said determined amount is below or above said predetermined threshold, or
   wherein the risk of getting a disease or an adverse event is predicted in said patient if said determined amount is below or above said predetermined threshold, or
   wherein a disease or an adverse event of said subject is monitored.
3. A method according to embodiment 1 and 2, wherein the level of PAM and/or its isoforms and/or fragments thereof is the total concentration of PAM and/or its isoforms and/or fragments thereof having at least 12 amino acids or the activity of PAM and/or its isoforms and/or fragments thereof in a sample of bodily fluid of said subject.
4. A method according to embodiments 1-3, wherein the activity of PAM and/or its isoforms and/or fragments thereof is selected from the group comprising the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.
5. A method according to embodiments 1-3, wherein the total concentration of PAM and/or its isoforms and/or fragments thereof having at least 12 amino acids is detected with an immunoassay.
6. A method according to embodiments 3-4, wherein the activity of PAM and/or its isoforms and/or fragments thereof is detected using a peptide-Gly as substrate.
7. A method according to embodiment 6, wherein the peptide-Gly substrate is selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromdin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.
8. A method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid of said subject according to embodiments 1-7, wherein the PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.
9. A method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid of said subject according to embodiments 1-8, wherein the risk of getting a disease of a subject is determined, wherein said subject is a healthy subject.
10. A method according to embodiment 9, wherein said disease is selected from the group of Alzheimer's disease, colorectal cancer and pancreatic cancer.
11. A method for determining the level of PAM and/or isoforms and/or fragments thereof in a bodily fluid sample using an assay, wherein said assay is comprising two binders that bind to two different regions of PAM, wherein the two binders are directed to an epitope of at least 5 amino acids, preferably at least 4 amino acids in length, wherein said two binders are directed to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).
12. A method for determining the activity of PAM and/or isoforms or fragments thereof in a bodily fluid sample of a subject comprising the steps
   contacting said sample with a capture-binder that binds specifically to active full-length PAM, its isoforms and/or active fragments thereof,
   separating PAM bound to said capture-binder,
   adding a substrate of PAM to said separated PAM, and
   quantifying PAM activity by measuring the conversion of the substrate of PAM.
13. A method for determining the activity of PAM and/or isoforms and/or fragments thereof in a bodily fluid sample of a subject comprising the steps
   contacting said sample with a substrate (peptide-Gly) of PAM for an interval of time at t=0 min and t=n+1 min
   detecting the reaction product (alpha-amidated peptide) of PAM in said sample at t=0 min and t=n+1 min, and
   quantifying the activity of PAM by calculating the difference of the reaction product between t=0 and t=n+1.
14. A method according to embodiment 13, wherein the peptide-Gly substrate is selected from the group comprising adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromdin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.
15. Use of antibodies for the determination of the level of PAM and/or its isoforms and/or fragments thereof, wherein said antibodies specifically bind to the sequences selected from the group of recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).
16. Kit for the determination of the level of PAM and/or its isoforms and/or fragments thereof, comprising one or more antibodies binding to PAM sequences selected from the group comprising recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24).

### FIGURE DESCRIPTION

FIG. 1: Schematic representation of PAM Isoform 1. Black bold arrows indicate cleavage-sites at double-basic amino-acids.

FIG. 2: Enzymatic reaction catalysed by PAM

FIG. 3: Representative calibration curve of recombinant PAM (ADM maturation activity [AMA].

FIG. 4: Frequency distribution (histogram) of AMA in self-reported healthy individuals (n=120)

FIG. 5: Correlation of AMA in matrix duplets (Li-heparin and serum) from self-reported healthy individuals (n=20)

FIG. 6A-L: Typical calibration curves of PAM sandwich immunoassays. A-J with recombinant PAM as calibration material. (A) solid phase: antibody directed to peptide 10 (SEQ ID No. 20), tracer: antibody directed to peptide 9 (SEQ ID No. 19); (B) solid phase: antibody directed to peptide 10 (SEQ ID No. 20), tracer: antibody directed to peptide 10 (SEQ ID No. 20); (C) solid phase: antibody directed to peptide 9 (SEQ ID No. 19), tracer: antibody directed to peptide 10 (SEQ ID No. 20); (D) solid phase: antibody directed to recombinant PAM (SEQ ID No. 10), tracer: antibody directed to recombinant PAM (SEQ ID No. 10); (E) solid phase: antibody directed to peptide 10 (SEQ ID No. 20), tracer: antibody directed to recombinant PAM (SEQ ID No. 10); (F) solid phase: antibody directed to peptide 13 (SEQ ID No. 23), tracer: antibody directed to peptide 10 (SEQ ID No. 20); (G) solid phase: antibody directed to peptide 14 (SEQ ID No. 24), tracer: antibody directed to peptide 13 (SEQ ID No. 23); (H) solid phase: antibody directed to recombinant PAM (SEQ ID No. 10), tracer: antibody directed to peptide 13 (SEQ ID No. 23); (I) solid phase: antibody directed to peptide 13 (SEQ ID No. 23), tracer: antibody directed to peptide 9 (SEQ ID No. 19); (J) solid phase: antibody directed to peptide 10 (SEQ ID No. 20), tracer: antibody directed to peptide 13 (SEQ ID No. 23). K and L with native PAM (EDTA-Plasma) as calibration material: (K) solid phase: antibody directed to peptide 14 (SEQ ID No. 24), tracer: antibody directed to peptide 13 (SEQ ID No. 23); (L) solid phase: antibody directed to peptide 10 (SEQ ID No. 20), tracer: antibody directed to peptide 13 (SEQ ID No. 23).

FIGS. 6 M-O: Enzyme capture assay (ECA)-(M) solid phase antibody directed to peptide 10 (SEQ ID No. 20); (N) solid phase antibody directed to full-length PAM (SEQ ID No. 10); (0) solid phase antibodies directed against peptide 7 (SEQ ID No. 17), peptide 8 (SEQ ID No. 18), peptide 9 (SEQ ID No. 19), peptide 13 (SEQ ID No. 23) and peptide 14 (SEQ ID No. 24) with recombinant PAM/heparin plasma used as sample.

FIG. 6 P: Correlation between PAM activity and PAM concentration in heparin samples from healthy volunteers (n=26; Spearman r=0.49, p=0.0109).

FIG. 7: Typical ADM-Gly dose/signal curve

FIG. 8: ADM maturation activity (PAM activity) in MPP-study (prediction of Alzheimer's disease)

FIG. 9: Kaplan-Meier-Plot (prediction of Alzheimer's disease [AD] in MPP-study)

FIG. 10: ADM maturation activity (PAM activity) in MPP-study (prediction of colorectal cancer [CRC])

FIG. 11: MR-proADM in MPP-study (prediction of colorectal cancer [CRC])

FIG. 12: Kaplan-Meier-Plot (prediction of colorectal cancer [CRC] in MPP-study)

FIG. 13: Diagnosis of pancreatic cancer in MPP-study

FIG. 14: Receiver operating curve (ROC Plot) of ADM maturation activity (PAM activity) for diagnosis of pancreatic cancer (MPP-study)

FIG. 15: Kaplan-Meier-Plot (prediction of all-cause mortality in MPP-study)

FIG. 16: Kaplan-Meier-Plot (prediction of cardiovascular mortality in MPP-study)

FIG. 17: Kaplan-Meier-Plot (prediction of heart failure in MPP-study)

FIG. 18: Kaplan-Meier-Plot (prediction of atrial fibrillation in MPP-study)

FIG. 19: ADM maturation activity (PAM activity) for diagnosis of prevalent Alzheimer's disease (AD)

FIG. 20: ADM maturation activity (PAM activity) for outcome prognosis of sepsis/septic shock in AdrenOSS-1 study (n=145 survivors; n=52 non-survivors)

FIG. 21: Kaplan-Meier-Plot of ADM maturation activity (PAM activity) for 28-day mortality (AdrenOSS-1 study)

FIG. 22: ADM maturation activity (PAM activity) in saliva of healthy subjects (n=5)

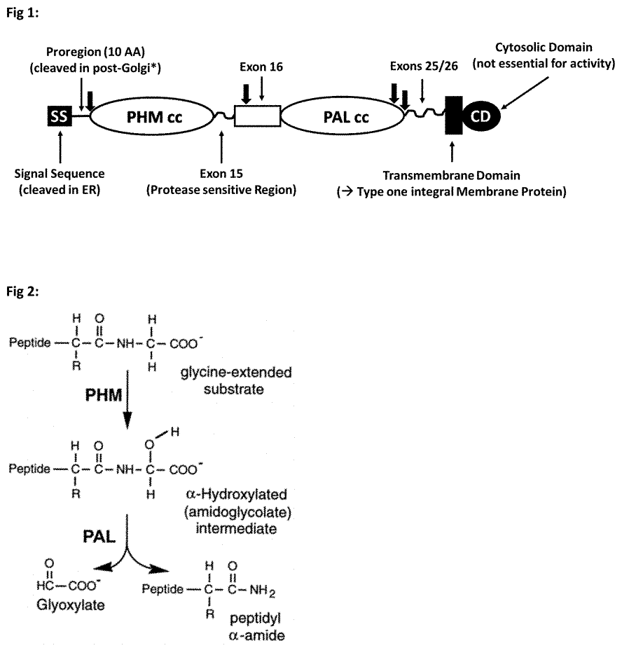

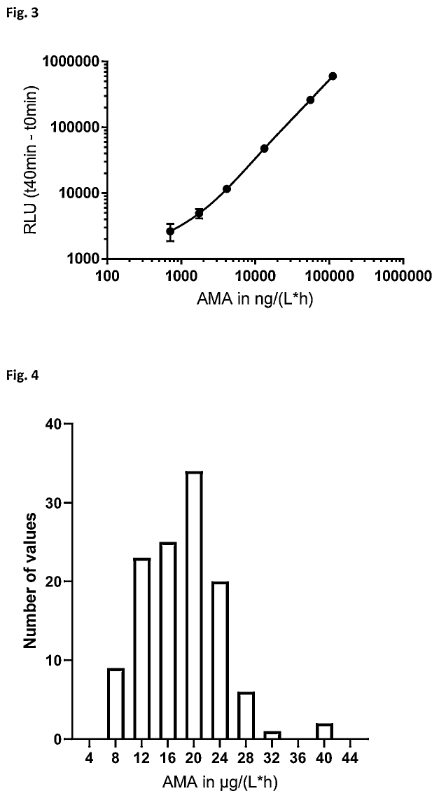

### EXAMPLES

### Example 1— Production of Recombinant PAM

PAM cDNA was synthesized according to Uniprot Accession No. P19021 encoding amino acids 21-834 of the PAM protein involving codon optimization for expression in mammalian cells. The signal sequence of PAM was replaced with human serum albumin signal sequence (MKWVTFISLLFLFSSAYSFR [SEQ ID No. 9]). At the C-terminus of PAM a hexa-histidine tag was added linked via a GS linker to PAM. The sequence of recombinant PAM (amino acids 21-834 of PAM without signal sequence and hexa-histidine tag) is shown in SEQ ID No. 10. The cDNA was cloned into an expression vector (plasmid DNA) using a 5′-NotI and a 3′ HindIII restriction site. The expression vector harboring the cDNA for PAM expression was replicated in- and prepared from E. coli. as a low-endotoxin preparation.

HEK-INV cells were transfected with the expression vector using INVect transfection reagents in serum free suspension culture. The transfection rate was controlled via co-transfection with a GFP-(green fluorescent protein) containing expression vector. Cultivation of cells was carried out in presence of valproic acid and Penicillin-Streptomycin at 37° C. and 5% CO₂. Cells were harvested via centrifugation when viability reached <60% (>2000 g, 30-45 min, 2-8° C.). Cell culture supernatant (CCS) was washed 5 times with 100 mM Tris/HCL pH 8.0 via tangential flow filtration (TFF, 30 kDa cut-off).

Purification of recombinant PAM included application of buffer exchanged CCS on a Q-sepharose fast flow resin (GE Healthcare) with a NaCl gradient (up to 2 M) elution. Amidating activity containing fractions were pooled and applied onto a Superdex 200 pg (GE Healthcare) size exclusion chromatography column with a 100 mM Tris/HCl, 200 mM NaCl, pH8.0 elution buffer. Amidating activity containing fractions were pooled, dialyzed against 100 mM Tris HCl, 200 mM NaCl, pH 8.0, sterile filtered (0.2 μm). Endotoxin load was determined by Charles River PTS Endosafe system and was below 5 EU/mL.

### Example 2—Production of Antibodies

Anti-PAM antibodies according to the present invention may be synthesised as follows:

PAM peptides for immunization were synthesized, see Table 1, (Peptides & Elephants, Hennigsdorf, Germany) with an additional C-terminal cysteine (if no cysteine is present within the selected PAM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio. Recombinant PAM was produced by InVivo Biotech Services, Hennigsdorf, as described in example 1.

TABLE 1
PAM immunization peptides

| Name (amino acid position*) | Sequence |
|---|---|
| Peptide 1 (aa 42-56) (SEQ ID No. 11) | CLGTTRPVVPIDSSD |
| Peptide 2 (aa 109-128) (SEQ ID No. 12) | CNMPSSTGSYWFCDEGTCTD |
| Peptide 3 (aa 168-180) (SEQ ID No. 13) | YGDISAFRDNNKD |
| Peptide 4 (aa 204-216) (SEQ ID No. 14) | SVDTVIPAGEKVV |
| Peptide 5 (aa 329-342) (SEQ ID No. 15) | CTQNVAPDMFRTIP |
| Peptide 6 (aa 291-310) (SEQ ID No. 16) | TGEGRTEATHIGGTSSDEMC |
| Peptide 7 (aa 234-244) (SEQ ID No. 17) | YRVHTHHLGKV |
| Peptide 8 (aa 261-276) (SEQ ID No. 18) | QSPQLPQAFYPVGHPV |
| Peptide 9 (aa 530-557) (SEQ ID No. 19) | RGDHVWDGNSFDSKFVYQQIGLGPIEED |
| Peptide 10 (aa 611-631) (SEQ ID No. 20) | EGPVLILGRSMQPGSDQNHFC |
| Peptide 11 (aa 562-579 (SEQ ID No. 21) | IDPNNAAVLQSSGKNLFY |
| Peptide 12 (aa 745-758) (SEQ ID No. 22) | NGKPHFGDQEPVQG |
| Peptide 13 (aa 669-687) (SEQ ID No. 23) | WGEESSGSSPLPGQFTVPH |
| Peptide 14 (aa 710-725) (SEQ ID No. 24) | CFKTDTKEFVREIKHS |
| Recombinant PAM | SEQ ID No. 10 |

*according to SEQ ID No. 1; amino acid (aa)

Balb/c mice were intraperitoneally (i.p.) injected with 100 μg recombinant PAM or 100 μg PAM-peptide-BSA-conjugates at day 0 (emulsified in TiterMax Gold Adjuvant), 100 μg and 100 μg at day 14 (emulsified in complete Freund's adjuvant) and 50 μg and 50 μg at day 21 and 28 (in incomplete Freund's adjuvant). The animal received an intravenous (i.v.) injection of 50 μg recombinant PAM at day 40 or 50 μg PAM-peptide-BSA-conjugates dissolved in saline at day 45. Three days later the mice were sacrificed and the immune cell fusion was performed.

Splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After one week, the HAT medium was replaced with HT Medium for three passages followed by returning to the normal cell culture medium.

The cell culture supernatants were primarily screened for recombinant PAM binding IgG antibodies two weeks after fusion. Therefore, recombinant PAM (SEQ ID No. 10) was immobilized in 96-well plates (100 μg/well) and incubated with 50 μl cell culture supernatant per well for 2 hours at room temperature. After washing of the plate, 50 μl/well POD-rabbit anti mouse IgG was added and incubated for 1 h at RT.

After a next washing step, 50 μl of a chromogen solution (3.7 mM o-phenylene-diamine in citrate/hydrogen phosphate buffer, 0.012% H₂O₂) were added to each well, incubated for 15 minutes at RT and the chromogenic reaction stopped by the addition of 50 μl 4N sulfuric acid. Absorption was detected at 490 mm.

The positive tested microcultures were transferred into 24-well plates for propagation. After retesting the selected cultures were cloned and re-cloned using the limiting-dilution technique and the isotypes were determined.

Antibodies raised against recombinant human PAM or PAM-peptides were produced via standard antibody production methods (Marx et al. 1997) and purified via Protein A. The antibody purities were >90% based on SDS gel electrophoresis analysis.

### Example 3—PAM Activity Assay

Human serum or Li-Heparin plasma from self-reported healthy volunteers was used as source of human native PAM. Each sample (200) was diluted two-fold in 100 mM Tris-HCl in duplicate. The amidation reaction was initiated by addition of 160 μl of PAM-reaction buffer (100 mM Tris-HCl, pH 7.5, 6.25 μM CuSO₄, 2.5 mM L-ascorbate, 125 μg/mL catalase, 62.5 μM amastatin, 250 μM leupeptin, 36 μg/mL synthetic ADM-Gly and 375 μg/mL NT-ADM antibody). Afterwards, 100 μl of each individual reaction of duplicated samples were combined and transferred into 20 μl of 200 mM EDTA to terminate the amidation reaction and to generate t=0 minutes reaction time-point followed by incubation at 37° C. for 40 minutes. Afterwards the non-terminated reactions were stopped with 10 μl of 200 mM EDTA. To determine the PAM activity, bio-ADM as reaction product was quantified in each sample using the Sphingotest® bio-ADM immunoassay (Weber et al. 2017). The amidation assay was calibrated using a 6-point calibration curve generated with human recombinant PAM of known activity. Samples and calibrators were treated in the same manner. Relative light units (RLU t40 min-t0 min) determined via Sphingotest® bio-ADM immunoassay for each sample were fitted against the RLU (t40 min-t0 min) of the calibrator to determine the PAM activity in the samples. PAM activity is described as “adrenomedullin maturation activity” (AMA) in μg bio-ADM formed per hour and L of sample.

A typical PAM calibration curve is shown in FIG. 3. The distribution of AMA in Li-Heparin samples from n=120 self-reported healthy volunteers are shown in FIG. 4. The median [IQR] of Li-Heparin AMA was 18.4 μg/(L*h) [13.5-21.9]. The 10^{th} and 90^{th} percentile was 10.5 and 24.2 μg/(L*h), respectively. The 2.5^{th}, 97.5^{th} and 99^{th} percentile was 8.1, 31.6 and 40.8 μg/(L*h). In addition, matched serum samples from n=20 subjects were measured and revealed a highly significant correlation (r=0.89; p<0.0001) (FIG. 5), although AMA values in serum were approximately 40% lower when compared to Li-Heparin.

### Example 4 PAM Immunoassays

Antibodies against recombinant PAM (SEQ ID No. 10) and PAM peptides (SEQ ID No. 11 to 24) were raised as described in example 1.

The technology used was a sandwich luminescence immunoassay, based on Akridinium ester labelling.

4.1. Labelled Compound (Tracer)

Purified antibodies (0.2 g/L) were labelled by incubation in 10% labelling buffer (500 mmol/L sodium phosphate, pH 8.0) with 1:5 mol/L ratio of MACN-acridinium-NHS-ester (1 g/L, InVent GmbH) for 20 min at 22° C. After adding 5% 1 mol/L Tris-HCl, pH 8.0, for 10 min, the respective antibody was separated from free label via CentriPure P10 columns (emp Biotech GmbH). The purified labelled antibody was diluted in 300 mmol/1 potassium phosphate, 100 mmol/1 NaCl, 10 mmol/1 Na-EDTA, 5 g/l Bovine Serum Albumin (pH 7.0). The final concentration was approximately 20 μg of labelled antibody per 150 μL.

4.2. Solid Phase

White polystyrene microtiter plates (Greiner Bio-One International AG) were coated (18 h at 20° C.) with the respective antibody (2 μg/0.2 mL per well 50 mmol/L Tris-HCl, 100 mmol/L NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried.

4.3 Calibration

The assay was calibrated, using dilutions of recombinant PAM as described in Example 1. The typical concentration range was within of 5-5,000 μg/mL.

4.4. Pam Immunoassays:

4.4.1. PAM-LIA

One-Step version: 50 μL, of samples/calibrators were pipetted into pre-coated microtiter plates. After adding 200 μL, of labelled antibody in buffer (300 mmol/L potassium phosphate, 100 mmol/L NaCl, 10 mmol/L Na-EDTA, 50 μmol/L amastatin, 100 μmol/L leupeptin, 0.1% bovine IgG, 0.02% mouse IgG, 0.5% BSA, pH 7.0), the microtiter plates were incubated for 20 h at 2-8° C. under agitation at 600 rpm. Unbound tracer was removed by washing 5 times (each 350 μL, per well) with washing solution (20 mmol/L PBS, 1 g/L Triton X-100, pH 7.4). Wellbound chemiluminescence was measured for 1 s per well by using the Centro LB 960 microtiter plate luminescence reader (Berthold Technologies).

Two-Step version: 50 μL of samples/calibrators were pipetted into pre-coated microtiter plates. After adding 200 μL of buffer (as described in one-step version), the microtiter plates were incubated for 15-20 h at 2-8° C. under agitation at 600 rpm. Unbound sample was removed by washing 4 times (each 350 μL per well) with washing solution with subsequent addition of 200 μl of tracer material and incubation of microtiter plates at room temperature for 2 h. Unbound tracer was removed by washing 4 times (each 350 μL per well) with washing solution. Well-bound chemiluminescence was measured for 1 s per well by using the Centro LB 960 microtiter plate luminescence reader (Berthold Technologies).

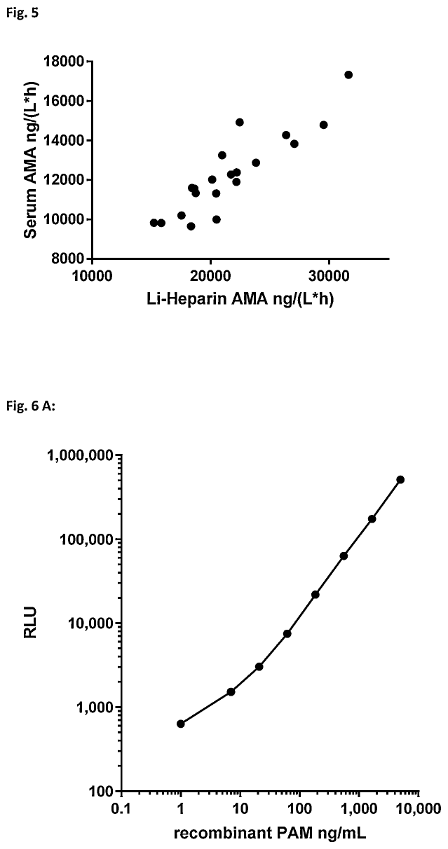

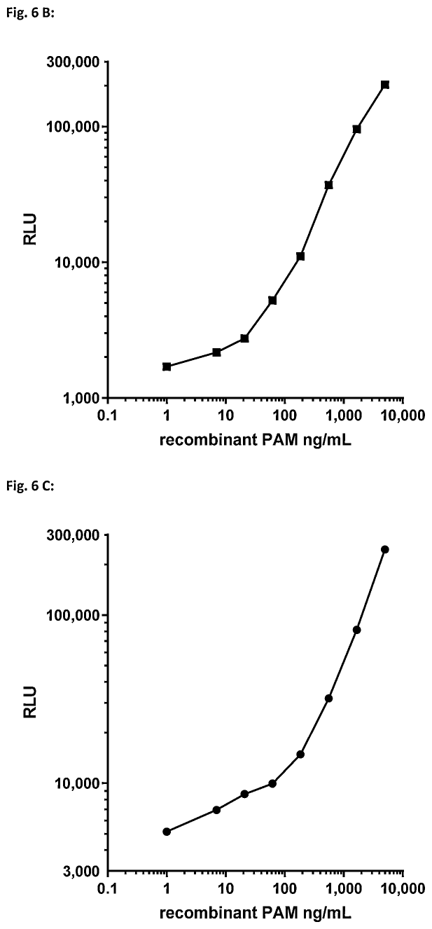

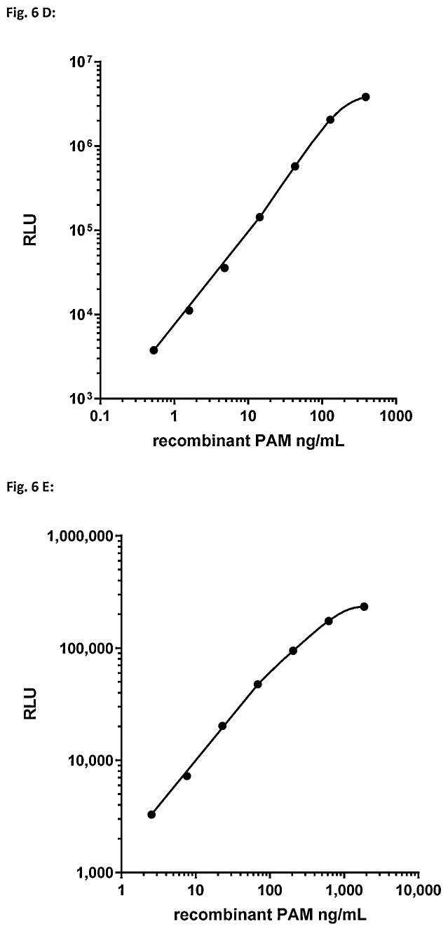

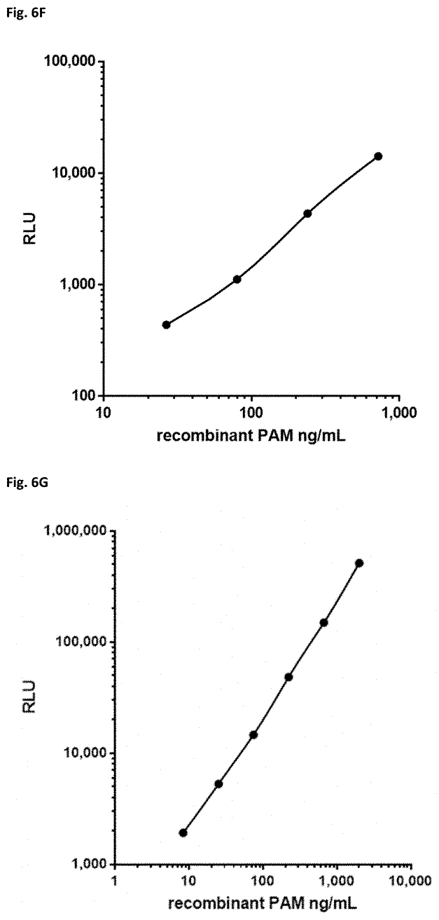

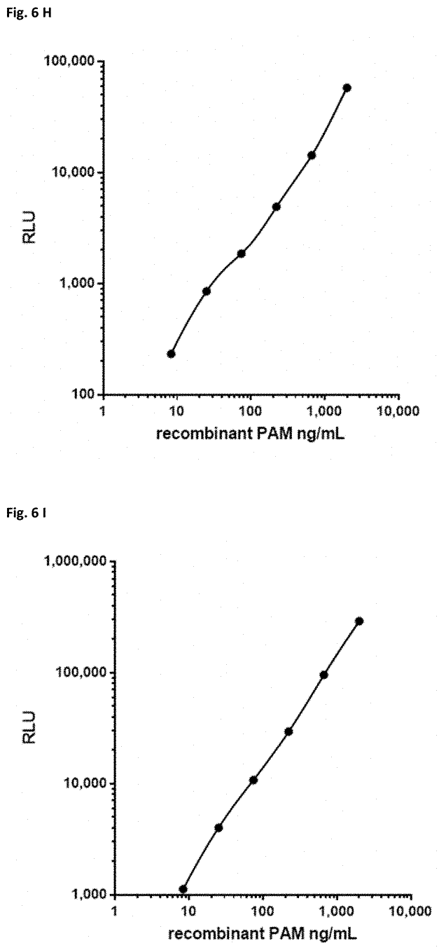

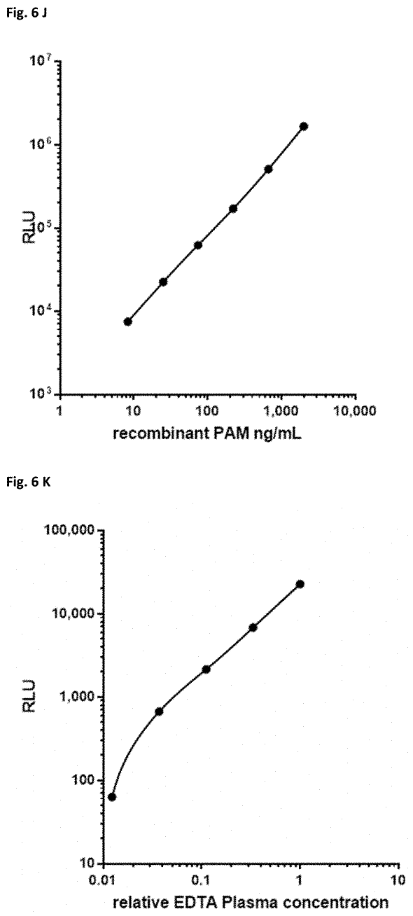

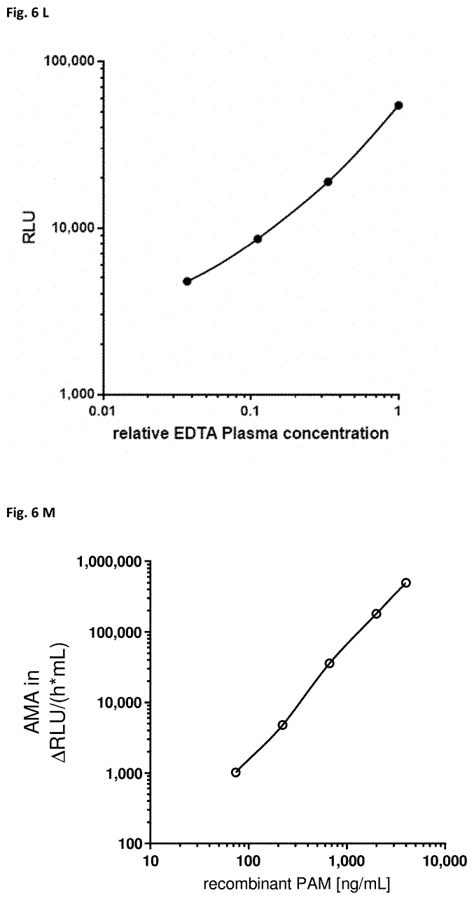

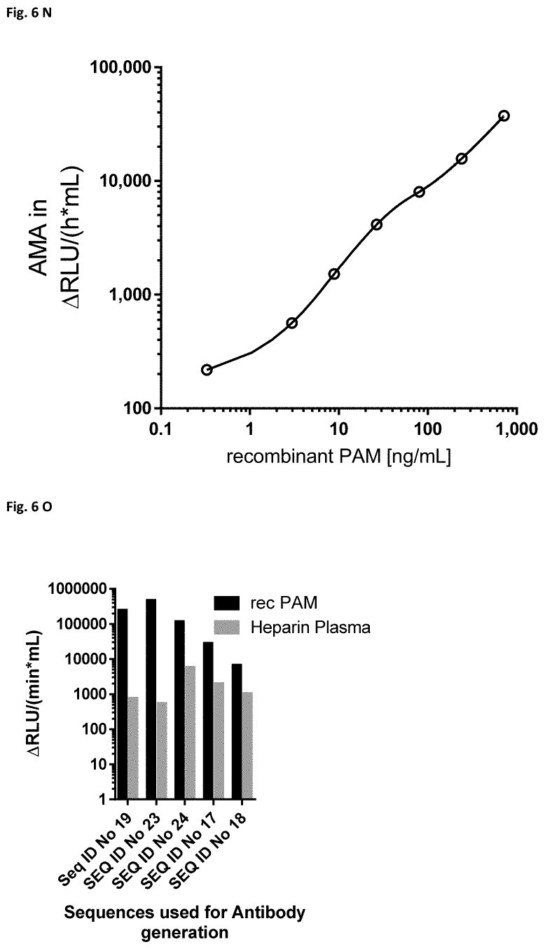

Results: Antibodies bound to the solid phase and labelled antibodies directed to the different PAM immunization peptides as well as full-length (recombinant) PAM (see example 2) were tested with recombinant PAM as well as blood samples. Exemplary standard curves for different antibody combinations are shown in FIGS. 6 (A-L). FIGS. 6 (A-J) with recombinant PAM as calibration material: (A) solid phase: antibody directed to peptide 10 (SEQ ID No. 20), tracer: antibody directed to peptide 9 (SEQ ID No. 19); (B) solid phase: antibody directed to peptide 10 (SEQ ID No. 20), tracer: antibody directed to peptide 10 (SEQ ID No. 20); (C) solid phase: antibody directed to peptide 9 (SEQ ID No. 19), tracer: antibody directed to peptide 10 (SEQ ID No. 20); (D) solid phase: antibody directed to recombinant PAM (SEQ ID No. 10), tracer: antibody directed to recombinant PAM (SEQ ID No. 10); (E) solid phase: antibody directed to peptide 10 (SEQ ID No. 20), tracer: antibody directed to recombinant PAM (SEQ ID No. 10); (F) solid phase: antibody directed to peptide 13 (SEQ ID No. 23), tracer: antibody directed to peptide 10 (SEQ ID No. 20); (G) solid phase: antibody directed to peptide 14 (SEQ ID No. 24), tracer: antibody directed to peptide 13 (SEQ ID No. 23); (H) solid phase: antibody directed to recombinant PAM (SEQ ID No. 10), tracer: antibody directed to peptide 13 (SEQ ID No. 23); (I) solid phase: antibody directed to peptide 13 (SEQ ID No. 23), tracer: antibody directed to peptide 9 (SEQ ID No. 19); (J) solid phase: antibody directed to peptide 10 (SEQ ID No. 20), tracer: antibody directed to peptide 13 (SEQ ID No. 23). FIGS. 6 (K and L) with native PAM (EDTA-Plasma) as calibration material: (K) solid phase: antibody directed to peptide 14 (SEQ ID No. 24), tracer: antibody directed to peptide 13 (SEQ ID No. 23); (L) solid phase: antibody directed to peptide 10 (SEQ ID No. 20), tracer: antibody directed to peptide 13 (SEQ ID No. 23). With all antibody combinations PAM was also detectable in human plasma and serum samples.

4.4.2. Enzyme Capture Assay (ECA) for the Detection of PAM Activity

Enzyme capture assays were established to detect the activity of PAM. 50 μL of samples/calibrators were pipetted into pre-coated microtiter plates (as described in 4.2.). After adding 200 μL of buffer (300 mmol/L potassium phosphate, 100 mmol/L NaCl, 50 μmol/L amastatin, 100 μmol/L leupeptin, 0.1% bovine IgG, 0.02% mouse IgG, 0.5% BSA, pH 7.0) the microtiter plates were incubated for 1 h at room temperature under agitation at 600 rpm. Unbound sample was removed by washing 4 times (each 350 μL per well) with washing solution with subsequent addition of 200 μl reaction buffer per well and incubation at 37° C. Reaction buffer including all components and final concentrations were as described in Example 3, with the exceptions that 100 μg/mL NT-ADM-antibody and 288 μg/mL ADM-Gly were used. Reaction was terminated at several time-points by transferring 10 μl of each individual reaction into 190 μl of EDTA containing buffer (300 mmol/L potassium phosphate, 100 mmol/L NaCl, 10 mmol/L Na-EDTA, 50 μmol/L amastatin, 100 μmol/L leupeptin, 0.1% bovine IgG, 0.02% mouse IgG, 0.5% BSA, pH 7.0). Terminated reactions were applied onto the Sphingotest® bio-ADM immunoassay for quantification of produced bio-ADM. A typical standard curve using an antibody directed to PAM immunization peptide 10 (SEQ ID No. 20) as solid phase is shown in FIG. 6 M. FIG. 6 N shows a typical standard curve using an antibody directed to full-length recombinant PAM (SEQ ID No. 10). Further antibodies directed to peptide 7 (SEQ ID No. 17), peptide 8 (SEQ ID No. 18), peptide 9 (SEQ ID No. 19), peptide 13 (SEQ ID No. 23) and peptide 14 (SEQ ID No. 24) were used as solid phase for the enzyme capture assay and a sample (250 μl) of recombinant PAM or heparin plasma was measured for PAM activity (FIG. 6 O). These results show, that the antibodies can be used to detect PAM activity in human samples using the ECA technique. PAM was also detectable in plasma and serum samples.

In a further step, PAM activity (as described in example 3) and PAM concentration using a PAM-LIA (solid phase antibody directed against full-length PAM, tracer antibody directed against peptide 13 [SEQ ID No. 23]) were determined in heparin samples from healthy volunteers (n=26). PAM activity and PAM concentration correlated significantly as shown in FIG. 6 P (Spearman r=0.49, p=0.0109).

### Example 5— ADM-Gly Immunoassay

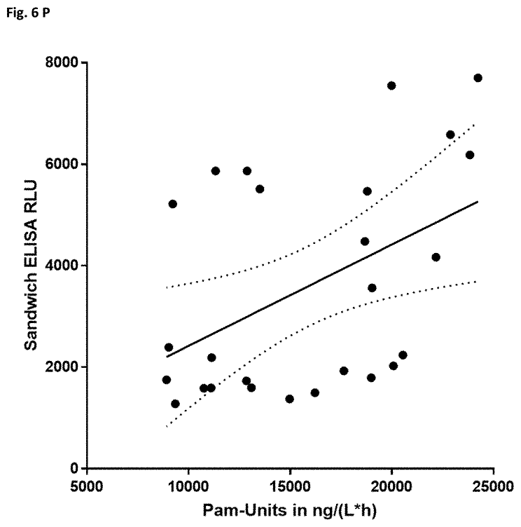

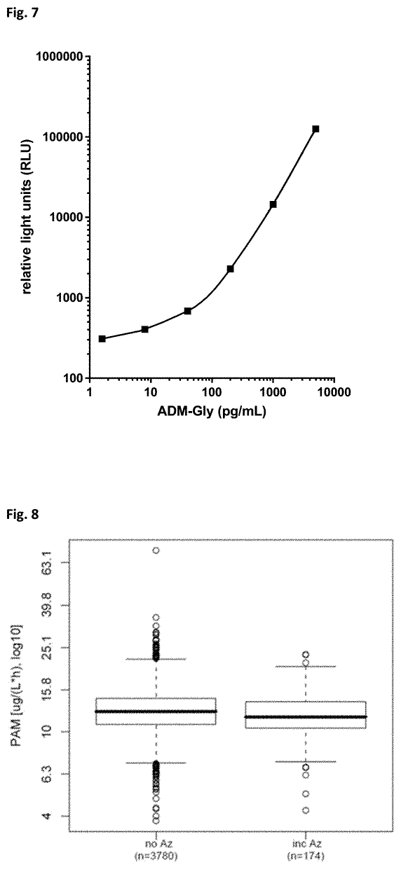

ADM-Gly was quantified as based on Weber et al. (Weber et al. 2017. JALM 2(2): 222-233) for bioactive ADM with the following modifications: the tracer-antibody used for ADM-Gly detection, labelled with MACN-acridinium-NHS, was directed to the C-terminal glycine of ADM-Gly. The assay was calibrated with synthetic ADM-Gly. The limit of detection (LOD) was 10 pg/mL of ADM-Gly. Cross-reactivity of antibody directed to the C-terminal glycine of ADM with bio-ADM was in the range between 6 and 50% in a concentration dependent manner. All determined ADM-Gly concentrations were corrected for cross-reactivity as follows: For each ADM-Gly quantification additional quantification of bio-ADM in corresponding samples was performed using the Sphingotest® bio-ADM immunoassay. The corresponding bio-ADM values were used to determine the signal (RLU) generated with the antibody directed to C-terminal glycine of ADM on a bio-ADM calibration curve. The determined signal (RLU) was used to calculate the false-positive ADM-Gly concentration (pg/mL) using the ADM-Gly calibration curve. This concentration was subtracted from the initially determined ADM-Gly concentration. A typical standard curve is shown in FIG. 7.

### Example 6— Prediction of Diseases in Healthy Subjects

6.1. Study Cohort

The Malmo Preventive Project (MPP) was funded in the mid-1970s to explore CV risk factors in general population, and enrolled 33,346 individuals living in Malmo (Fedorowski et al. 2010. Eur Heart J 31: 85-91). Between 2002 and 2006, a total of 18,240 original participants responded to the invitation (participation rate, 70.5%) and were screened including a comprehensive physical examination and collection of blood samples (Fava et al. 2013. Hypertension 2013; 61: 319-26). The re-examination in MPP is in the present study regarded as the baseline. Subjects with prior CVD at baseline were excluded.

An informed consent was obtained from all participants and the Ethical Committee of Lund University, Lund, Sweden, approved the study protocol.

A commercial fully automated homogeneous time-resolved fluoro-immunoassay was used to measure MR-proADM in plasma (BRAHMS MR-proADM KRYPTOR; BRAHMS GmbH, Hennigsdorf, Germany) (Caruhel et al. 2009. Clin Biochem. 42 (7-8): 725-8).

Bio-ADM was measured as described by Weber et al. 2017 (Weber et al. 2017. JAMA 2(2): 222-233). AMA was determined in 4942 serum samples from MPP as described in example 3. Each sample was measured in duplicate. Samples, controls and calibrators were treated in the same manner. Baseline clinical characteristics of AMA after stratification to Quartiles is shown in table 2.

TABLE 2
Baseline clinical characteristics according to quartile (Q) of AMA at baseline of subjects analysed

| | Q1 | Q2 | Q3 | Q4 | |
|---|---|---|---|---|---|
| | (n = 1235) | (n = 1236) | (n = 1236) | (n = 1235) | P |
| AMA in μg/(L*h) (SD) | 9.416 (1.21) | 11.66 (0.46) | 13.39 (0.57) | 17 (3) | N/A |
| AMA range | 3.8-10.86 | 10.86-12.47 | 12.47-14.47 | 14.47-72.15 | N/A |
| Age in years (SD) | 68.97 (6.18) | 69.16 (6.28) | 69.34 (6.38) | 70.45 (6.07) | <0.0001 |
| Current smoking, n (%) | 188 (15.2) | 217 (17.6) | 255 (20.6) | 287 (23.2) | <0.0001 |
| Systolic blood pressure | 144 (19.77) | 145.1 (19.83) | 144.8 (20.33) | 147.6 (21.34) | <0.0002 |
| in mmHg (SD) | | | | | |
| Diastolic blood pressure | 82.83 (10.12) | 84.04 (10.83) | 83.12 (10.61) | 84.45 (11.51) | 0.0041 |
| mmHg (SD) | | | | | |
| Diabetes Mellitus, n (%) | 166 (13.4) | 127 (10.3) | 113 (9.1) | 127 (10.3) | 0.0043 |
| Glucose in mmol/L (SD) | 6.024 (1.95) | 5.78 (1.21) | 5.794 (1.37) | 5.753 (1.28) | 0.0299 |

N/A: not applicable

Statistical analysis: Values are expressed as means and standard deviations, medians and interquartile ranges (IQR), or counts and percentages as appropriate. Group comparisons of continuous variables were performed using the Kruskal-Wallis test. Biomarker data were log-transformed. Cox proportional-hazards regression was used to analyze the effect of risk factors on survival in uni- and multivariable analyses. The assumptions of proportional hazard were tested for all variables. For continuous variables, hazard ratios (HR) were standardized to describe the HR for a biomarker change of one IQR. 95% confidence intervals (CI) for risk factors and significance levels for chi-square (Wald test) are given. The predictive value of each model was assessed by the model likelihood ratio chi-square statistic. The concordance index (C index) is given as an effect measure. It is equivalent to the concept of AUC adopted for binary outcome. For multivariable models, a bootstrap corrected version of the C index is given. Survival curves plotted by the Kaplan-Meier method were used for illustrative purposes. To test for independence of PAM from clinical variables we used the likelihood ratio chi-square test for nested models. All statistical tests were 2-tailed and a two-sided p-value of 0.05 was considered for significance.

6.2. Prediction of Alzheimer's Disease

3954 samples with information about dementia diagnosis refilling efficiency in hemodialysed CKD patients were selected (n=174 with incident AD). Information about dementia diagnoses was requested from the Swedish National Patient Register (SNPR). The diagnoses in the register were collected according to different revisions of the International Classification of Diseases (ICD) codes 290, 293 (ICD-8), 290, 331 (ICD-9) or FOO, F01, F03, G30 (ICD-10). Since 1987, SNPR includes all in-patient care in Sweden and, in addition, contains data on outpatient visits including day surgery and psychiatric care from both private and public caregivers recorded after 2000. All-cause dementia was diagnosed according to the criteria of the Diagnostic and Statistical Manual of Mental Disorders (DSM)-III revised edition, whilst the DSM-IV criteria were applied for the Alzheimer's disease and vascular dementia diagnoses. Diagnoses were validated by a thorough review of medical records as well as neuroimaging data when available. A research physician assigned the final diagnosis for each patient and a geriatrician specialized in cognitive disorders was consulted in unresolved cases. The PAM activity (AMA) was determined as described in example 3. AMA in the MPP cohort is shown in FIG. 8: AMA in patients developing AD over time (incident AD, n=174) are significantly lower compared to the non-AD group (p=0.01).

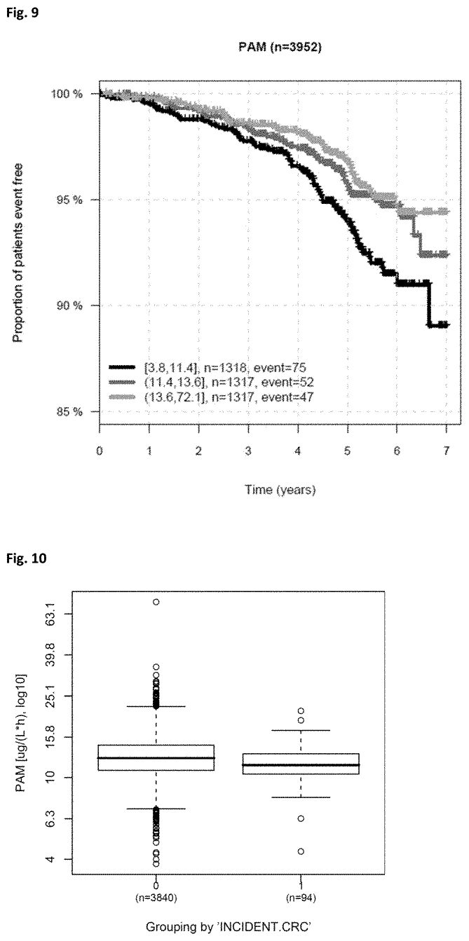

Reduced serum AMA strongly predicts Alzheimer's disease with a Hazard Ratio (HR) of 0.74 (CI 0.6-0.88; p<0.001) and a HR of 0.72 (CI 0.6-0.85) when adjusted for age (table 3). FIG. 9 shows a Kaplan-Meier Plot for the prediction of Alzheimer's disease using AMA (prevalent AD cases were excluded from the analysis). The lowest tertile is associated with the highest risk of getting AD.

Furthermore, AMA as a predictor of AD was independent from bio-ADM concentrations. Both markers contribute to AD prediction. While the C-Index for AMA alone is 0.571 (CI 0.525-0.616; Chi² 10.97) the C-index for both combined markers, i.e. AMA and bio-ADM is 0.595 (Chi² 18.96; p<0.0001).

Moreover, AMA in combination with bio-ADM and MR-proADM concentrations further improve the prediction of incident Alzheimer. While MR-proADM alone had no predictive value for AD, the combination of AMA, bio-ADM and MR-proADM showed a C-index of 0.622 (Chi² 26.73; p=0.00001).

TABLE 3
Prediction of Alzheimer's disease

| | Hazard Ratio | | | |
|---|---|---|---|---|
| Biomarker | (HR) (CI) | p-Value | C-Index (CI) | Chi² |
| AMA | 0.72 (0.6-0.85) | p < 0.001 | 0.571 (0.525-0.616) | 10.97 |
| AMA, bio-ADM | | p < 0.0001 | 0.595 | 18.96 |

6.3. Prediction of Colorectal Cancer (CRC)

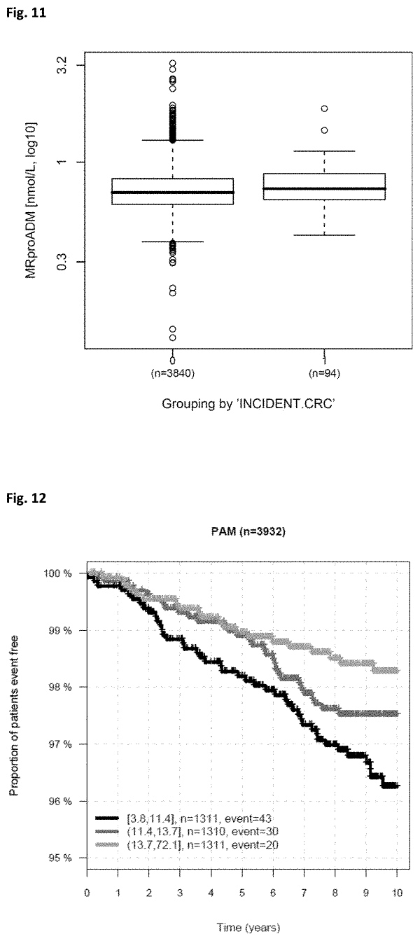

AMA of subjects with and without incident CRC is shown in FIG. 10. The AMA in patients developing CRC over time (n=93) are significantly lower compared to the non-CRC group (p=0.0008; Kruskal-Wallis). In contrast, the MR-proADM concentrations in patients developing CRC over time are higher compared to the non-CRC group (p=0.023) as shown in FIG. 11.

Results for the single markers and marker combinations are shown in Table 3. Reduced serum AMA (age-adjusted) strongly predicts development of CRC with a Hazard Ratio (HR) of 0.68 (p<0.0001). FIG. 12 shows a Kaplan-Meier Plot for the prediction of CRC with AMA (prevalent cases were excluded from the analysis). The lowest tertile is associated with the highest risk of CRC development (p<0.005).

Increased MR-proADM concentrations predict development of CRC with a HR of 1.36 p<0.05). The highest quartile is associated with the highest risk of CRC development (p=0.051).

While bio-ADM concentrations were not predictive for development of CRC, a combination of bio-ADM and AMA showed an improved CRC prediction (see table 4). In addition, a combination of AMA and MR-proADM further improved the prediction of CRC development.

In summary, reduced AMA values predict development of CRC. Increased MR-proADM concentrations also predict development of CRC. A combination of AMA with bio-ADM or MR-proADM enhances the predictive value for CRC.

TABLE 4
Prediction of colorectal cancer

| | Hazard Ratio | | | |
|---|---|---|---|---|
| Biomarker | (HR) (CI) | p-Value | C-Index (CI) | Chi² |
| AMA | 0.68 (0.6-0.85) | p < 0.00001 | 0.588 (0.535-0.641) | 8.51 |
| AMA, bioADM | | p < 0.002 | 0.598 | 12.48 |
| MR-proADM | 1.36 (1.08-1.72) | p < 0.05 | 0.587 (0.532-0.642) | 6.27 |
| AMA, MR-proADM | | p < 0.0005 | 0.612 | 16.51 |

6.4. Prediction of Pancreatic Cancer

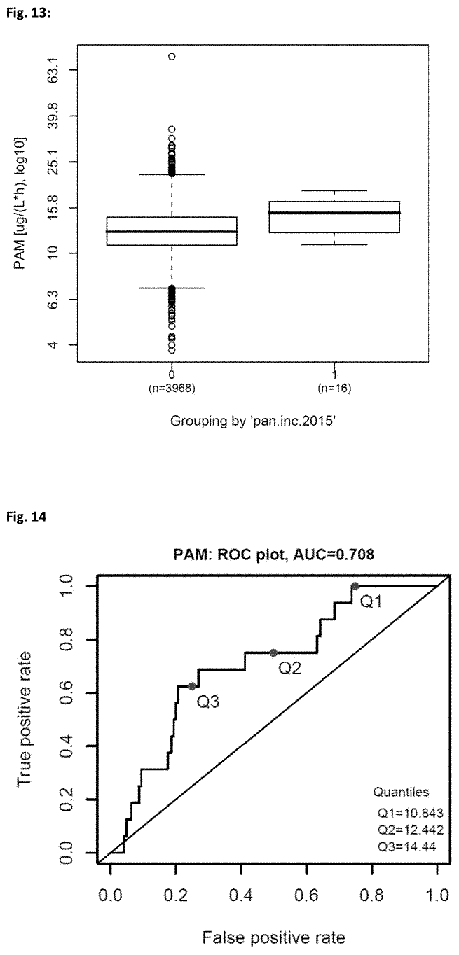

Moreover, AMA is increased in incident pancreatic cancer compared to subjects without pancreatic cancer (p<0.005) (FIG. 13). AMA strongly predicts pancreatic cancer with an Odds Ratio (OR) of 0.44 (CI 0.33-0.58). The respective receiver operating curve (ROC plot) for AMA is shown in FIG. 14 and revealed an AUC of 0.71.

6.5. Prediction of all-Cause and Cardiovascular Mortality

Mortality analyses were performed in 4942 samples with information about death and cardiovascular events from the MPP cohort. Information about cardiovascular events and diagnoses was requested from the Swedish National Patient Register (SNPR). The diagnoses in the register were collected according to different revisions of the International Classification of Diseases (ICD) codes. Since 1987, SNPR includes all in-patient care in Sweden and, in addition, contains data on outpatient visits including day surgery and psychiatric care from both private and public caregivers recorded after 2000. The PAM activity (AMA) was determined as described in example 3. Within of the total set of 4942 serum samples from the MPP study cohort 1361 subjects died (all-cause mortality) during follow-up period of 12.8 years. From the total number of 1361 death-events 480 events where accounted to as cardiovascular mortality.

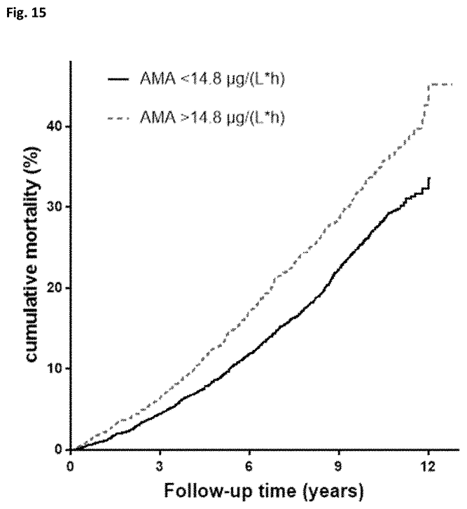

Elevated serum AMA strongly predicts all-cause mortality with a Hazard Ratio (HR) of 1.354 (CI 1.197-1.531; p<0.0001) (Table 5). The predictive value of AMA was independent of the common cardiovascular risk factors (age, gender, blood-pressure, body-mass index, antihypertensive medication, low- and high-density lipoproteins and history of diabetes). FIG. 15 shows a Kaplan-Meier Plot for the prediction of All-cause mortality using AMA. High AMA is associated with increased risk of mortality.

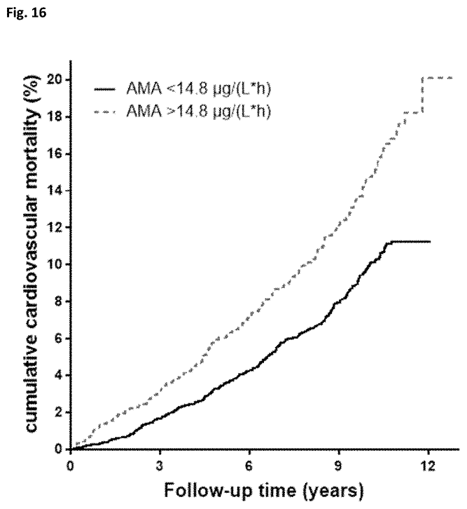

Elevated serum AMA strongly predicts cardiovascular mortality with a Hazard Ratio (HR) of 1.6 (CI 1.3-1.969; p<0.0001) (Table 5). The predictive value of AMA was independent of the common cardiovascular risk factors (age, gender, blood-pressure, body-mass index, antihypertensive medication, low- and high-density lipoproteins and history of diabetes). FIG. 16 shows a Kaplan-Meier Plot for the prediction of cardiovascular mortality using AMA. High AMA is associated with increased risk of cardiovascular mortality.

TABLE 5
Prediction of all-cause and cardiovascular mortality by PAM activity

| | Q1 | Q2 |
|---|---|---|
| | (n = 3707) | (n = 1235) |
| AMA in μg/(L*h) (SD) | 11.49 (1.82) | 17 (3) |
| AMA range | 3.8-14.47 | 14.47-72.15 |

All-Cause Mortality

| | | |
|---|---|---|
| Number of Events | 943 | 418 |
| Logrank Hazard Ratio | (ref) | 1.354 (1.197-1.531) |
| (95% CI) | | |
| Chi² | | 26.8 |
| p-value | | <0.0001 |

Cardiovascular mortality

| | | |
|---|---|---|
| Number of Events | 314 | 166 |
| Logrank Hazard Ratio | (ref) | 1.6 (1.3-1.969) |
| (95% CI) | | |
| Chi² | | 24.38 |
| p-value | | <0.0001 |

6.6. Prediction of Cardiovascular Disorders

Cardiovascular disorder analyses were performed in 4942 samples with information about death- and cardiovascular events from the MPP cohort. Information about cardiovascular events and diagnoses was requested from the Swedish National Patient Register (SNPR). The diagnoses in the register were collected according to different revisions of the International Classification of Diseases (ICD) codes. Since 1987, SNPR includes all in-patient care in Sweden and, in addition, contains data on outpatient visits including day surgery and psychiatric care from both private and public caregivers recorded after 2000. The PAM activity (AMA) was determined as described in example 3. Within of the total set of 4942 serum samples from the MPP study cohort 278 subjects developed heart failure (incident heart failure) and 633 subjects developed atrial fibrillation (incident atrial fibrillation) during follow-up period of 12.8 years.

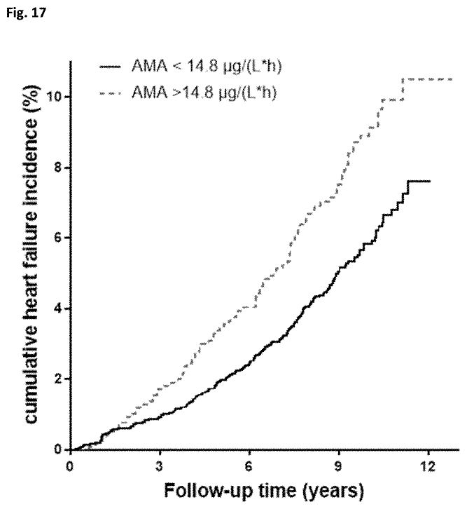

Elevated serum AMA strongly predicts incident heart failure (83 prevalent HF cases were excluded from the analyses) with a Hazard Ratio (HR) of 1.537 (CI 1.169-2.021; p<0.0007) (Table 6). FIG. 17 shows a Kaplan-Meier Plot for the prediction of All-cause mortality using AMA. High AMA is associated with increased risk of getting heart failure.

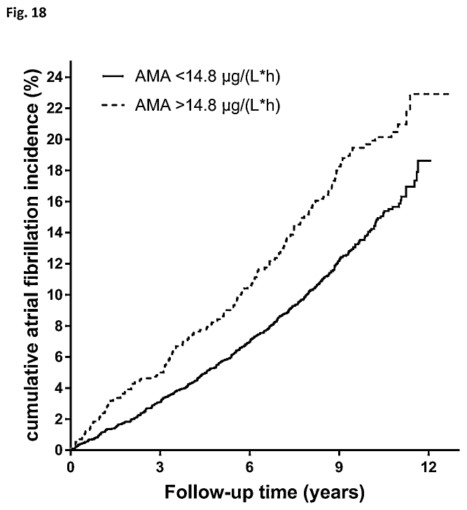

Elevated serum AMA strongly predicts incident atrial fibrillation (267 prevalent AF cases were excluded from the analyses) with a Hazard Ratio (HR) of 1.459 (CI 1.214-1.752; p<0.0001) (Table 6). FIG. 18 shows a Kaplan-Meier Plot for the prediction of All-cause mortality using AMA. High AMA is associated with increased risk of getting heart failure.

TABLE 6
Prediction of cardiovascular disorders

| | |
|---|---|
| Q1 | Q2 |
| (n = 3707) | (n = 1119) |

Heart failure

| | | |
|---|---|---|
| Number of Events | 186 | 92 |
| Logrank Hazard Ratio | (ref) | 1.537 (1.169-2.021) |
| (95% CI) | | |
| Chi² | | 11.56 |
| p-value | | =0.0007 |

Atrial fibrillation

| | Q1 | Q2 |
|---|---|---|
| | (n = 3534) | (n = 1141) |
| Number of Events | 436 | 197 |
| Logrank Hazard Ratio | (ref) | 1.459 (1.214-1.752) |
| (95% CI) | | |
| Chi² | | 19.59 |
| p-value | | <0.0001 |

### Example 7— Diagnosis of Diseases

7.1. Diagnosis of Alzheimer's Disease

Serum samples from 27 individuals with diagnosed Alzheimer's disease were obtained from InVent Diagnostica GmbH. The AD diagnosis is based on cognitive tests (CERAD, DemTec, MMST and Clock-Drawing test) as well as on MRI (Magnetic resonance imaging) and CT-scans. As controls, 67 serum samples from self-reported healthy volunteers were used. AMA was detected as described in example 3.

As shown in FIG. 19 patients from the AD-cohort showed significantly lower serum AMA when compared to the control-cohort (n=67; p<0.0001).

7.2. Diagnosis of Cardiovascular and Metabolic Disorders

In the total set of 4942 serum samples from the MPP study cohort, 267 cases of prevalent atrial fibrillation, 83 cases of prevalent chronic heart failure and 533 cases of prevalent diabetes were present.

Significant elevation of serum AMA (p<0.0001) was observed in prevalent atrial fibrillation (mean AMA: 13.92 AMA-Units, n=267) when compared to individuals free of prevalent atrial fibrillation (mean AMA: 12.8 AMA-Units, n=4675).

Significant elevation of serum AMA (p=0.0019) was observed in prevalent chronic heart failure (mean AMA: 14.31 AMA-Units, n=83) when compared to individuals free of prevalent heart failure (mean AMA: 12.84 AMA-Units, n=4859).

Significant reduction of serum AMA (p=0.0035) was observed in prevalent diabetes (mean AMA: 12.69 AMA-Units, n=533) when compared to individuals free of prevalent diabetes (mean AMA: 12.89 AMA-Units, n=4409).

### Example 8— Prognosis and Monitoring

8.1. Study Cohort AdrenOSS-1

AdrenOSS-1 was a European prospective observational study. Twenty-four centers in five countries (France, Belgium, The Netherlands, Italy, and Germany) contributed to the trial achievement of 583 enrolled patients (recruited from June 2015 to May 2016). The study protocol was approved by the local ethics committees and was conducted in accordance with the Declaration of Helsinki. The study enrolled patients aged 18 years and older who were (1) admitted to the ICU for sepsis or septic shock or (2) transferred from another ICU in the state of sepsis and septic shock within less than 24 h after admission. Included patients were stratified by severe sepsis and septic shock based on definitions for sepsis and organ failure from 2001 (Levy et al. 2003. 2001 SCCM/ESICM/ACCP/ATS/SIS International Sepsis Definitions Conference. Crit Care Med. 31(4):1250-6). The term “sepsis” refers to the updated definition of Sepsis-3 (Singer et al. 2016 The Third International Consensus Definitions for Sepsis and Septic Shock (Sepsis-3). JAMA. 315(8):801-10). Patients were treated according to local practice, and treatments as well as procedures were registered. The primary endpoint was 28-day mortality. Secondary endpoints concerned organ failure (as defined by the Sequential Organ Failure Assessment [SOFA] score) and organ support, vasopressor/inotrope use, fluid balance, and use of renal replacement therapy (RRT).

Upon admission, demographics (age, sex), body mass index, presence of septic shock, type of ICU admission, organ dysfunction scores (SOFA, Acute Physiologic Assessment and Chronic Health Evaluation II [APACHE II]), origin of sepsis, pre-existing comorbidities (i.e., treated within the last year), past medical history, laboratory values, and organ support were recorded, and blood was drawn for measurement of bio-ADM and other markers. After patient enrolment, the following data were collected daily during the first week: SOFA score, antimicrobial therapies, fluid balance, ventilation status, Glasgow Coma Scale score, central venous pressure, need for RRT, invasive procedures for sepsis control, and vasopressor/inotrope treatment. Moreover, discharge status and mortality were recorded on day 28 after ICU admission.

Blood for the central laboratory was sampled within 24 h after ICU admission and on day 2 (mean 47 h, SD 9 h) after the first sample. Samples were subsequently processed and stored at −80° C. The PAM activity (AMA) was measured in n=197 plasma samples, randomly selected from AdrenOSS-I cohort as described in example 3.

8.2. Outcome Prognosis in Sepsis

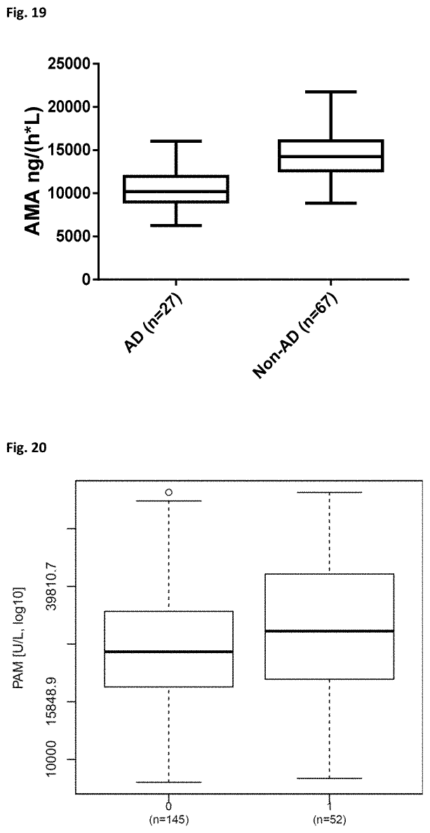

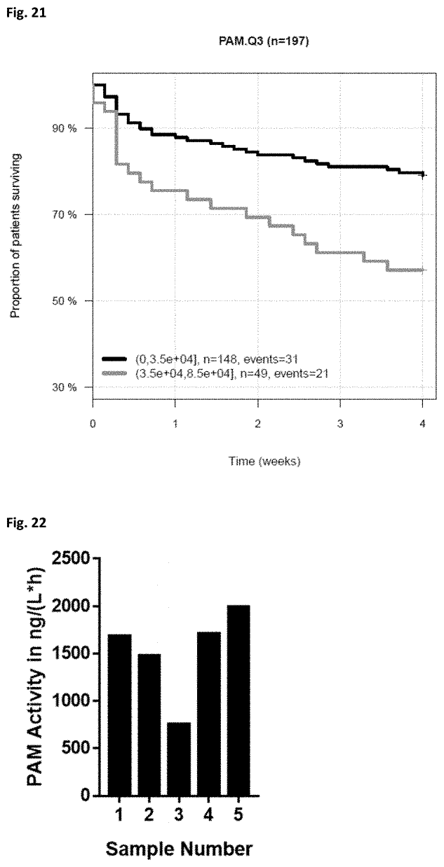

The AMA in the AdrenOSS-I cohort as shown in FIG. 20 revealed a significantly higher AMA in the non-survivor group compared to the surviving group (p<0.05). High plasma AMA strongly predict 28-day mortality with a HR of 1.41 (p<0.05). FIG. 21 shows a Kaplan-Meier Plot for the prediction of 28-day mortality in patients with sepsis and septic shock.

In addition, the ADM-Gly concentrations in the AdrenOSS-I cohort, also revealed a significantly higher concentration in non-survivors compared to survivors (p<0.0001). High ADM-Gly concentrations strongly predict 28-day mortality with a HR of 2.29 (p<0.005).

The outcome for the 28-day mortality for the single biomarkers AMA and ADM-Gly is shown in table 7. A cut-off for AMA and ADM-Gly concentration, respectively, was chosen to result in an equal sensitivity of 80.4%, while the specificity was 21.7% for AMA and 38.5% for ADM-Gly, respectively. When both markers were combined by, i.e. a ratio, the specificity for 28-day survival was increased to 43.4%. Furthermore, the Odds ratio (OR) was 1.13 and 2.56 for PAM and ADM Gly, respectively, while the combination of both markers resulted in an increased OR of 3.14.

TABLE 7
Cross-tables for the evaluation of 28 day mortality outcome.

| [μg/(L*h)] | 28 Day Mortality | 28 Day Survival | |
|---|---|---|---|
| AMA > 17.1 | 41 | 112 | PPV: 26.8% |
| AMA < 17.1 | 10 | 31 | NPV: 75.6% |
| Prevalence: 26.3% | Sensitivity: 80.4% | Specificity: 21.7% | OR: 1.13 |
| [pg/mL] | 28 Day Mortality | 28 Day Survival | 15 |
| ADM-Gly > 140 | 41 | 88 | PPV: 31.8% |
| ADM-Gly < 140 | 10 | 55 | NPV: 84.6% |
| Prevalence: 26.3% | Sensitivity: 80.4% | Specificity: 38.5% | OR: 2.56 |
| AMA/ADM-Gly | | | |
| ratio | 28 D Mortality | 28 D Survival | |
| AMA/ADM- | 41 | 81 | PPV: 33.6% |
| Gly < 132 | | | |
| AMA/ADM- | 10 | 62 | NPV: 86.1% |
| Gly > 132 | | | |
| Prevalence: 26.3% | Sensitivity: 80.4% | Specificity: 43.4% | OR: 3.14 |

9. Determination of PAM Activity in Human Saliva

Saliva was collected from 5 self-reported healthy subjects in separate sterile tubes. PAM activity in human saliva samples was tested as described in example 3. PAM activity could be measured in saliva samples (range from around 700 to 2000 μg/(L*h) (FIG. 22) and was approximately 10-times lower compared to plasma samples.

SEQUENCES

```
SEQ ID NO: 1-Prepro-PAM isoform 1 AS 1-973
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGDSEH
              910 920 930 940 950
KLETSSGRVL GRFRGKGSGG LNLGNFFASR KGYSRKGFDR LSTEGSDQEK
              960 970
EDDGSESEEE YSAPLPALAP SSS
SEQ ID NO: 2-Prepro-PAM isoform 2 AS 1-868
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQDFH MEEALDWPGV
              410 420 430 440 450
YLLPGQVSGV ALDPKNNLVI FHRGDHVWDG NSFDSKFVYQ QIGLGPIEED
              460 470 480 490 500
TILVIDPNNA AVLQSSGKNL FYLPHGLSID KDGNYWVTDV ALHQVFKLDP
              510 520 530 540 550
NNKEGPVLIL GRSMQPGSDQ NHFCQPTDVA VDPGTGAIYV SDGYCNSRIV
              560 570 580 590 600
QFSPSGKFIT QWGEESSGSS PLPGQFTVPH SLALVPLLGQ LCVADRENGR
              610 620 630 640 650
IQCFKTDTKE FVREIKHSSF GRNVFAISYI PGLLFAVNGK PHFGDQEPVQ
              660 670 680 690 700
GFVMNFSNGE IIDIFKPVRK HFDMPHDIVA SEDGTVYIGD AHTNTVWKFT
              710 720 730 740 750
LTEKLEHRSV KKAGIEVQEI KEAEAVVETK MENKPTSSEL QKMQEKQKLI
              760 770 780 790 800
KEPGSGVPVV LITTLLVIPV VVLLAIAIFI RWKKSRAFGD SEHKLETSSG
              810 820 830 840 850
RVLGRFRGKG SGGLNLGNFF ASRKGYSRKG FDRLSTEGSD QEKEDDGSES
              860
EEEYSAPLPA LAPSSS
SEQ ID No.: 3-Prepro-PAM isoform 3 AS (amino acids 829-896 of
SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKDS EHKLETSSGR VLGRFRGKGS
              860 870 880 890 900
GGLNLGNFFA SRKGYSRKGF DRLSTEGSDQ EKEDDGSESE EEYSAPLPAL
    905
APSSS
SEQ ID No. 4-Prepro-PAM isoform 4 (amino acids 829-914 of
SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKGK GSGGLNLGNF FASRKGYSRK
              860 870 880 890
GFDRLSTEGS DQEKEDDGSE SEEEYSAPLP ALAPSSS
SEQ ID No. 5-Prepro-PAM Isoform 5 (Isoform 1 with an additional aa in
position 896)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNCEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QIKQKIIKIP GSGVPVVLIT TLLVIPVVVL LAIAIFIRWK KSRAFGADSE
              910 920 930 940 950
HKLETSSGRV LGRFRGKGSG GLNLGNFFAS RKGYSRKGFD RLSTEGSDQE
              960 970
KEDDGSESEE EYSAPLPALA PSSS
SEQ ID No. 6-Prepro-PAM Isoform 6 (amino acids 897-914 of SEQ ID
No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVISIQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHID MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGGKGS
              910 920 930 940 950
GGLNLGNFFA SRKGYSRKGF DRLSTEGSDQ EKEDDGSESE EEYSAPLPAL
APSSS
SEQ ID No. 7-PHM subunit of PAM
                10 20 30 40 50
FKETTRPFSN ECLGTTRPVV PIDSSDFALD IRMPGVTPKQ SDTYFCMSMR
                60 70 80 90 100
IPVDEEAFVI DFKPRASMDT VHHMLLFGCN MPSSTGSYWF CDEGTCTKDA
              110 120 130 140 150
NILYAWARNA PPTRLPKGVG FRVGGETGSK YFVLQVHYGD ISAFRDNNKD
              160 170 180 190 200
CSGVSLHLTR LPQPLIAGMY LMMSVDTVIP AGEKVVNSDI SCHYKNYPMH
              210 220 230 240 250
VFAYRVHTHH LGKVVSGYRV RNGQWTLIGR QSPQLPQAFY PVGHPVDVSF
              260 270 280 290 300
GDLLAARCVF TGEGRTEATH IGGTSSDEMC NLYIMYYMEA KHAVSFMTCT
              310 320 330 340 350
QNVAPDMFRT IPPEANIPIP VKSDMVMMHE HHKETEYKDK IPLLQQPKRE
              360 370 380 390 400
EEEVLDQGDF YSLLSKLLGE REDVVHVHKY NPTEKAESES DLVAEIANVV
              410 420 430 440 450
QKKDLGRSDA REGAEHERGN
              460
PPPGEGTWEP EHTG
SEQ ID No. 8-PAL subunit of PAM
                10 20 30 40 50
DFHMEEALDW PGVYLLPGQV SGVALDPKNN LVIFHRGDHV WDGNSFDSKF
                60 70 80 90 100
VYQQIGLGPI EEDTILVIDP NNAAVLQSSG KNLFYLPHGL SIDKDGNYWV
              110 120 130 140 150
TDVALHQVFK LDPNNKEGPV LILGRSMQPG SDQNHFCQPT DVAVDPGTGA
              160 170 180 190 200
IYVSDGYCNS RIVQFSPSGK FITQWGEESS GSSPLPGQFT VPHSLALVPL
              210 220 230 240 250
LGQLCVADRE NGRIQCFKTD TKEFVREIKH SSFGRNVFAI SYIPGLLFAV
              260 270 280 290 300
NGKPHFGDQE PVQGFVMNFS NGEIIDIFKP VRKHFDMPHD IVASEDGTVY
              310 320
IGDAHTNTVW KFTLTEKLEH RSV
SEQ ID No. 9-signal sequence human serum albumin
                10 20
MKWVTFISLL FLFSSAYSFR
SEQ ID No. 10-Sequence of recombinant human PAM
                10 20 30 40 50
SPLSVFKRFK ETTRPFSNEC LGTTRPVVPI DSSDFALDIR MPGVTPKQSD
                60 70 80 90 100
TYFCMSMRIP VDEEAFVIDF KPRASMDTVH HMLLFGCNMP SSTGSYWFCD
              110 120 130 140 150
EGTCTDKANI LYAWARNAPP TRLPKGVGFR VGGETGSKYF VLQVHYGDIS
              160 170 180 190 200
AFRDNNKDCS GVSLHLTRLP QPLIAGMYLM MSVDTVIPAG EKVVNSDISC
              210 220 230 240 250
HYKNYPMHVF AYRVHTHHLG KVVSGYRVRN GQWTLIGRQS PQLPQAFYPV
              260 270 280 290 300
GHPVDVSFGD LLAARCVFTG EGRTEATHIG GTSSDEMCNL YIMYYMEAKH
              310 320 330 340 350
AVSFMTCTQN VAPDMFRTIP PEANIPIPVK SDMVMMHEHH KETEYKDKIP
              360 370 380 390 400
LLQQPKREEE EVLDQGDFYS LLSKLLGERE DVVHVHKYNP TEKAESESDL
              410 420 430 440 450
VAEIANVVQK KDLGRSDARE GAEHERGNAI LVRDRIHKFH RLVSTLRPPE
              460 470 480 490 500
SRVFSLQQPP PGEGTWEPEH TGDFHMEEAL DWPGVYLLPG QVSGVALDPK
              510 520 530 540 550
NNLVIFHRGD HVWDGNSFDS KFVYQQIGLG PIEEDTILVI DPNNAAVLQS
              560 570 580 590 600
SGKNLFYLPH GLSIDKDGNY WVTDVALHQV FKLDPNNKEG PVLILGRSMQ
              610 620 630 640 650
PGSDQNHFCQ PTDVAVDPGT GAIYVSDGYC NSRIVQFSPS GKFITQWGEE
              660 670 680 690 700
SSGSSPLPGQ FTVPHSLALV PLLGQLCVAD RENGRIQCFK TDTKEFVREI
              710 720 730 740 750
KHSSFGRNVF AISYIPGLLF AVNGKPHFGD QEPVQGFVMN FSNGEIIDIF
              760 770 780 790 800
KPVRKHFDMP HDIVASEDGT VYIGDAHTNT VWKFTLTEKL EHRSVKKAGI
              810
EVQEIKEAEA VVGS
SEQ ID No. 11-Peptide 1 (aa 42-56 of PAM SEQ ID No. 1)
                10
CLGTTRPVVP IDSSD
SEQ ID No. 12-Peptide 2 (aa 109-128 of PAM SEQ ID No. 1)
                10
CNMPSSTGSY WFCDEGTCTD
SEQ ID No. 13-Peptide 3 (aa 168-180 of PAM SEQ ID No. 1)
                10
YGDISAFRDN NKD
SEQ ID No. 14-Peptide 4 (aa 204-216 of PAM SEQ ID No. 1)
                10
SVDTVIPAGE KVV
SEQ ID No. 15-Peptide 5 (aa 329-342 of PAM SEQ ID No. 1)
                10
CTQNVAPDMF RTIP
SEQ ID No. 16-Peptide 6 (aa 291-310 of PAM SEQ ID No. 1)
                10 20
TGEGRTEATH IGGTSSDEMC
SEQ ID No. 17-Peptide 7 (aa 234-244 of PAM SEQ ID No. 1)
                10
YRVHTHHLGK V
SEQ ID No. 18-Peptide 8 (aa 261-276 of PAM SEQ ID No. 1)
                10
QSPQLPQAFY PVGHPV
SEQ ID No. 19-Peptide 9 (aa 530-557 of PAM SEQ ID No. 1)
                10 20
RGDHVWDGNS FDSKFVYQQI GLGPIEED
SEQ ID No. 20-Peptide 10 (aa 611-631 of PAM SEQ ID No. 1)
                10 20
EGPVLILGRS MQPGSDQNHF C
SEQ ID No. 21-Peptide 11 (aa 562-579 of PAM SEQ ID No. 1)
                10
IDPNNAAVLQ SSGKNLFY
SEQ ID No. 22-Peptide 12 (aa 745-758 of PAM SEQ ID No. 1)
                10
NGKPHFGDQE PVQG
SEQ ID No. 23-Peptide 13 (aa 669-687 of PAM SEQ ID No. 1)
                10
WGEESSGSSP LPGQFTVPH
SEQ ID No. 24-Peptide 14 (aa 710-725 of PAM SEQ ID No. 1)
                10
CFKTDTKEFV REIKHS
```

1. A method for diagnosis or prognosis of a disease in a patient and/or predicting a risk of getting a disease or an adverse event in a patient and/or monitoring a disease or an adverse event in a patient, comprising:
determining the level of peptidylglycine alpha-amidating monooxygenase (PAM) and/or its isoforms and/or fragments thereof in a sample of bodily fluid of said patient,
wherein the disease in said patient is selected from dementia, cardiovascular disorders, kidney diseases, cancer, inflammatory or infectious diseases and/or metabolic diseases, and
wherein the adverse event is selected from a cardiac event, a cardiovascular event, a cerebrovascular event, a cancer, diabetes, infections, serious infections, sepsis-like systemic infections, sepsis and death due to all causes.

2. A method for diagnosis or prognosis of a disease in a patient and/or predicting a risk of getting a disease or an adverse event in a patient and/or monitoring a disease or adverse event in a patient by determining the level of peptidylglycine alpha-amidating monooxygenase (PAM) and/or its isoforms and/or fragments thereof in a sample of bodily fluid of said patient, the method comprising:
determining the level of PAM and/or its isoforms and/or fragments thereof in a sample of bodily fluid of said patient, and
comparing said determined amount to a predetermined threshold,
wherein said patient is diagnosed as having a disease if said determined amount is below or above said predetermined threshold, or
wherein an outcome of a disease is prognosticated if said determined amount is below or above said predetermined threshold, or
wherein the risk of getting a disease or an adverse event is predicted in said patient if said determined amount is below or above said predetermined threshold, or
wherein a disease or an adverse event of said patient is monitored.

3. A method according to claim 1, wherein the level of PAM and/or its isoforms and/or fragments thereof is the total concentration of PAM and/or its isoforms and/or fragments thereof having at least 12 amino acids or the activity of PAM and/or its isoforms and/or fragments thereof in said sample of bodily fluid of said patient.

4. A method according to claim 3, wherein the activity of PAM and/or its isoforms and/or fragments thereof is selected from the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

5. A method according to claim 3, wherein the total concentration of PAM and/or its isoforms and/or fragments thereof having at least 12 amino acids is detected with an immunoassay.

6. A method according to claim 3, wherein the activity of PAM and/or its isoforms and/or fragments thereof is detected using a peptide-Gly as substrate.

7. A method according to claim 6, wherein the peptide-Gly substrate is selected from adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.

8. A method for diagnosis or prognosis of a disease in a patient and/or predicting a risk of getting a disease or adverse event in a patient and/or monitoring a disease or adverse event in a patient by determining the level of PAM and/or its isoforms and/or fragments thereof in a sample of bodily fluid of said patient according to claim 1, wherein the PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

9. A method for diagnosis or prognosis of a disease in a patient and/or predicting a risk of getting a disease or adverse event in a patient and/or monitoring a disease or adverse event in a patient, comprising: determining the level of PAM and/or its isoforms and/or fragments thereof in a sample of bodily fluid of said patient according to claim 1, wherein the risk of getting a disease of a patient is determined, wherein said patient is a healthy patient.

10. A method according to claim 9, wherein said disease is selected from Alzheimer's disease, colorectal cancer and pancreatic cancer.

11. A method for determining the level of PAM and/or isoforms and/or fragments thereof in a bodily fluid sample using an assay, wherein said assay comprises two binders that bind to two different regions of PAM, wherein the two binders are directed to an epitope of at least 5 amino acids, preferably at least 4 amino acids in length, wherein said two binders are directed to an epitope comprised within the following sequences of PAM: peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) peptide 14 (SEQ ID No. 24) and recombinant PAM (SEQ ID No. 10).

12. A method for determining the activity of PAM and/or isoforms or fragments thereof in a bodily fluid sample of a patient comprising:
contacting said sample with a capture-binder that binds specifically to active full-length PAM, its isoforms and/or active fragments thereof,
separating PAM bound to said capture-binder,
adding a substrate of PAM to said separated PAM, and
quantifying PAM activity by measuring the conversion of the substrate of PAM.

13. A method for determining the activity of PAM and/or isoforms and/or fragments thereof in a bodily fluid sample of a patient, comprising:
contacting said sample with a substrate (peptide-Gly) of PAM for an interval of time at t=0 min and t=n+1 min, detecting the reaction product (alpha-amidated peptide) of PAM in said sample at t=0 min and t=n+1 min, and
quantifying the activity of PAM by calculating the difference of the reaction product between t=0 and t=n+1.

14. A method according to claim 13, wherein the peptide-Gly substrate is selected from adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.

15. (canceled)

16. A kit for the determination of the level of PAM and/or its isoforms and/or fragments thereof, comprising one or more antibodies binding to PAM sequences selected from the group comprising recombinant PAM (SEQ ID No. 10), peptide 1 (SEQ ID No. 11), peptide 2 (SEQ ID No. 12), peptide (SEQ ID No. 13), peptide 4 (SEQ ID No. 14), peptide 5 (SEQ ID No. 15), peptide 6 (SEQ ID No. 16), peptide 7 (SEQ ID No. 17), peptide 8 (SEQ ID No. 18), peptide 9 (SEQ ID No. 19), peptide 10 (SEQ ID No. 20), peptide 11 (SEQ ID No. 21), peptide 12 (SEQ ID No. 22), peptide 13 (SEQ ID No. 23) and peptide 14 (SEQ ID No. 24.

17. A method according to claim 2, wherein the level of PAM and/or its isoforms and/or fragments thereof is the total concentration of PAM and/or its isoforms and/or fragments thereof having at least 12 amino acids or the activity of PAM and/or its isoforms and/or fragments thereof in said sample of bodily fluid of said patient.

18. A method according to claim 17, wherein the activity of PAM and/or its isoforms and/or fragments thereof is selected from the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

19. A method according to claim 17, wherein the total concentration of PAM and/or its isoforms and/or fragments thereof having at least 12 amino acids is detected with an immunoassay.

20. A method according to claim 17, wherein the activity of PAM and/or its isoforms and/or fragments thereof is detected using a peptide-Gly as substrate.

21. A method according to claim 20, wherein the peptide-Gly substrate is selected from adrenomedullin (ADM), adrenomedullin-2, intermedin-short, pro-adrenomedullin N-20 terminal peptide (PAMP), amylin, gastrin-releasing peptide, neuromedin C, neuromedin B, neuromedin S, neuromedin U, calcitonin, calcitonin gene-related peptide (CGRP) 1 and 2, islet amyloid polypeptide, chromogranin A, insulin, pancreastatin, prolactin-releasing peptide (PrRP), cholecystokinin, big gastrin, gastrin, glucagon-like peptide 1 (GLP-1), pituitary adenylate cyclase-activating polypeptide (PACAP), secretin, somatoliberin, peptide histidine methionine (PHM), vasoactive intestinal peptide (VIP), gonadoliberin, kisspeptin, MIF-1, metastin, neuropeptide K, neuropeptide gamma, substance P, neurokinin A, neurokinin B, peptide YY, pancreatic hormone, deltorphin I, orexin A and B, melanotropin alpha (alpha-MSH), melanotropin gamma, thyrotropin-releasing hormone (TRH), oxytocin and vasopressin.
